(19) United States
(12) Patent Application Publication
(10) Pub. No.: US 2025/0076302 A1
(43) Pub. Date: Mar. 6, 2025

(54) DETECTING THE PRESENCE OF URINARY STONE DISEASE USING A PLURALITY OF URINARY METABOLITES

(71) Applicant: The Cleveland Clinic Foundation, Cleveland, OH (US)

(72) Inventors: Aaron W. Miller, Chesterland, OH (US); Jose Agudelo, Beachwood, OH (US)

(21) Appl. No.: 18/740,776

(22) Filed: Jun. 12, 2024

Related U.S. Application Data
(60) Provisional application No. 63/507,665, filed on Jun. 12, 2023.

(51) Int. Cl. G01N 33/68; G01N 33/70; G01N 33/94

(52) U.S. Cl. CPC G01N 33/6806; G01N 33/6848; G01N 33/70; G01N 33/94; G01N 2570/00; G01N 2800/34

(57) ABSTRACT

The present invention relates to systems, kits, and methods for identifying subjects with increased levels (e.g., in a urine sample) of at least two urine metabolites selected from: butanal; 2-Hydroxy-1,3-dimethoxy-8,9-methylenedioxycoumestan; 6-Methylmercaptopurine; Dimethyl-L-arginine; N-butanoyl-lhomoserine lactone; Hexanoylglycine; Methyl propenyl ketone; Ferulate; 2-Oxoarginine; and 2-Hydroxyestradiol-3-methyl ether, as well as methods of determining if a subject has or is at risk of urinary stone disease based on such urine metabolites. In certain embodiments, the urinary tract of a subject with elevated levels of at least two of the urine metabolites is imaged (e.g., to generate an image showing the size, location, or number of urinary stones present).

The present application claims priority to U.S. Provisional application Ser. No. 63/507,665, filed Jun. 12, 2023, which is herein incorporated by reference in its entirety.

This invention was made with government support under DK136517 awarded by the National Institutes of Health. The government has certain rights in the invention.

### FIELD OF THE INVENTION

The present invention relates to systems, kits, and methods for identifying subjects with increased levels (e.g., in a urine sample, or sample derived from a urine sample) of at least two urine metabolites selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether, as well as methods of determining if a subject has or is at risk of urinary stone disease based on such urine metabolites. In certain embodiments, the urinary tract of a subject with elevated levels of at least two of the urine metabolites is imaged (e.g., to generate an image showing the size, location, or number of urinary stones present).

### BACKGROUND

Urinary stone disease (USD) afflicts 1 in 11 Americans and incurs an annual expenditure of approximately $10 billion in healthcare costs¹. USD is a chronic disease with long asymptomatic periods and high recurrence rate². More than 90% of cases involve calcium-based stones (CBS)³, most of which are labeled as idiopathic⁴. While a few studies have examined the urinary metabolome in the context of USD^{5, 6}, many questions remain about the interactions between urinary metabolites and lithogenesis.

Urine is a source of nearly 5000 metabolites, collectively called the metabolome, many of which have been associated with disease⁷. Urinary metabolites can come from the host, microbiome⁷, as well as from the diet or biochemical interactions, and can strongly influence biomineralization⁸. Similarities in urinary chemistry abnormalities exist among patients with different urinary stone compositions. For instance, low urinary citrate is associated with the development of uric acid or CBS⁹. Moreover, a prospective study of stone formers with and without radiographic stones demonstrated that USD patients with different stone compositions exhibited a lower urinary concentration of osteopontin¹⁰.

### SUMMARY OF THE INVENTION

The present invention relates to systems, kits, and methods for identifying subjects with increased levels (e.g., in a urine sample, or sample derived from a urine sample) of at least two urine metabolites selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether (or at least two urine metabolites selected from those recited in Table 5, Appendix A), as well as methods of determining if a subject has or is at risk of urinary stone disease based on such urine metabolites. In certain embodiments, the urinary tract of a subject with elevated levels of at least two of the urine metabolites is imaged (e.g., to generate an image showing the size, location, or number of urinary stones present).

In some embodiments, provided herein are compositions, kits, and systems comprising: a) a sample from a subject (e.g., human subject) having: urinary stone disease, or who is suspected of having urinary stone disease, or who has a recurrent episode of urinary stone disease, and b) at least two stable isotope labeled urine metabolites selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether; and/or selected from at least two stable isotope labeled urine metabolites selected from Table 5 (Appendix A).

In particular embodiments, the stable isotope is selected from ²H, ¹³C, and ¹⁵N. In other embodiments, the sample comprises a urine sample. The compositions, kits, and systems further comprise: c) un-labelled versions of the at least two urine metabolites. In additional embodiments, the sample further comprises a stable isotope labeled third, fourth, fifth, sixth, seventh, eighth, ninth, or tenth stable isotope labeled urinary metabolite from the recited group.

In certain embodiments, provided herein are methods of performing an activity based on a level of at least two urine metabolites in a urine sample from a subject (e.g., human subject) comprising: a) determining, or receiving information regarding, the level of the at least two urine metabolites in, or from, a urine sample from a subject, wherein the at least two urine metabolites are selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether; and/or selected from Table 5 (Appendix A); and b) identifying an increased level of the at least two urine metabolites in the sample compared to corresponding control values, and performing at least one of the following activities: i) imaging the urinary tract of the subject and generating an image that shows the size and/or number and/or position of at least one urinary stone; ii) treating the subject with an agent or procedure that treats urinary stone disease; iii) generating and/or transmitting a report that: A) displays the level of the at least two urinary metabolites, and B) indicates that the subject is in need of: A) the imaging, and/or B) the agent or procedure that treat urinary stone disease; and iv) characterizing the subject as having urinary stones, recurrence of urinary stones, urinary stone disease, recurrence of urinary stone disease, having recurrent stone activity, or an elevated risk for urinary stones or urinary stone disease.

In further embodiments, the at least two urine metabolites is at least three, at least four, at least five, at least six, at least seven, at least eight, at least nine, or all ten, of the urine metabolites. In particular embodiments, the imaging is selected from: computed tomography (CT), ultrasound, x-ray, and KUB x-ray; and/or B) the urinary stone disease therapeutic is selected from: thiazide, a potassium supplement, a magnesium supplement, and a calcium supplement; and/or C) wherein the procedure comprises removal of at least one urinary stone from the subject. In additional embodiments, the subject has, or is suspected of having, urinary stone disease, or wherein the subject has, or is suspected of having, recurrent urinary stone disease. In further embodiments, determining the level comprises the use of mass spectrometry with chromatography. In additional embodiments, the urine sample comprises a spot urine sample.

In some embodiments, the report comprises a paper report or an electronic report; and/or wherein the receiving information comprises receiving the report, wherein the receiving the report is optionally via: 1) the mail system, 2) email, or 3) via a LAN of a hospital or clinic. In additional embodiments, the transmitting the report comprises: 1) mailing the reporting through the mail system, 2) emailing the report over the internet, or 3) sending the report through a local area network (LAN) or a hospital or clinic. In additional embodiments, the corresponding control values are derived from samples from the general public or from a group known to not have urinary stone disease or be at risk for urinary stone disease. In further embodiments, the determining comprises detecting the at least two urinary metabolites with an analytical device selected from: a mass spectrometer, NMR spectrometer, and a UV/Vis spectrometer.

In some embodiments, provided herein are methods of treatment and/or imaging comprising: a) identifying a subject as having increased levels of at least two urinary metabolites compared to corresponding reference values, wherein the at least two urinary metabolites are selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether; and/or selected from Table 5 (Appendix A); and b) performing at least one of the following activities: i) treating the subject with a therapeutic or procedure that treats urinary stone disease, and/or ii) imaging the urinary tract of the subject and generating an image that shows the size and/or number and/or position of at least one urinary stone, and optionally generating a written plan of care tailored to the size, number, and/or position of the at least one urinary stone.

In additional embodiments, the receiving information comprises receiving a report, wherein the receiving the report is optionally via: 1) the mail system, 2) email, or 3) via a LAN of a hospital or clinic. In other embodiments, the at least two urine metabolites is at least three, at least four, at least five, at least six, at least seven, at least eight, at least nine, or all ten, of the urine metabolites. In particular embodiments, wherein: i) the urinary stone disease therapeutic is selected from: thiazide, a potassium supplement, a magnesium supplement, and a calcium supplement, and ii) wherein the written plan comprises instructions for therapeutic treatment, expulsive therapy, and/or surgical intervention to remove the at least one urinary stone, and/or iii) the imaging is selected from: computed tomography (CT), ultrasound, x-ray, and KUB x-ray.

In additional embodiments, the subject (e.g., human subject): i) has, or is suspected of having, urinary stone disease, has recurrence of urinary stones, iii) has recurrence of urinary stone disease, iv) has recurrent stone activity, or v) has an elevated risk for urinary stones or urinary stone disease. In further embodiments, the corresponding control values are derived from samples from the general public or from a group known to not have urinary stone recurrence or active stone disease.

In some embodiments, provide herein are methods of detecting the level of at least two urinary metabolites in, or from a urine sample from a subject comprising: a) obtaining a urine sample, wherein the urine sample is from a human subject suspected of having urinary stone disease or recurrence of urinary stone disease; and b) treating the urine sample, or derivative thereof, under conditions such that the level of the at least two urinary metabolites is determined, wherein the at least two urinary metabolites are selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether; and/or selected from Table 5 (Appendix A), and c) optionally imaging the urinary tract of the subject, and further optionally generating an image that shows the size, location, or number of urinary stones. In additional embodiments, the treating comprises adding to the sample at least two stable isotope labeled urine metabolites selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether.

### DESCRIPTION OF THE FIGURES

The patent or application file contains at least one drawing executed in color. Copies of this patent or patent application publication with color drawings will be provided by the Office upon request and payment of the necessary fee.

FIG. 1. Distribution of metabolite presence from untargeted metabolomics runs. A) Metabolites extracted from CaOx & CaPhos stones; B) Metabolites extracted from the urine of patients; Data indicate that most metabolites in stones and urine are broadly distributed among the population. C) Metabolomic profile of the kidney stones and urine, stratified by stone composition and radiographic recurrence status. Metabolites are only listed if they comprised >0.1% of the metabolome and were given putative ID.

FIG. 2. Whole metabolome comparisons. PCoA plot based on a Binomial distance matrix of urine and kidney stone samples (A), CaOx and CaPhos samples (B), and the urine of active and non-active SF (C). PERMANOVA was used to statistically compare metabolome composition between groups. Differential abundance analysis to identify specific metabolites enriched in CaOx or CaPhos stones (D), or active or non-active SF (E). For differential abundance analysis, an FDR-corrected permuted Welch's t-test was conducted on mass (stone) or creatinine (urine) normalized metabolite levels. Red dots indicate metabolites that were significantly different between groups, whereas black dots are insignificant metabolites. Listed are the total number of metabolites in the dataset, along with the number of metabolites enriched in either group. Statistical analyses were conducted in Vegan.

FIG. 3. Workflow for a prospective clinical study.

FIG. 4. Algorithm used to validate the 14 metabolites identified in the Example as drivers differentiating the urinary metabolome of active and non-active SF.

FIG. 5. Comparison of samples vs. negative controls. A) Binomial distribution analysis show that samples are significantly different than negative controls. Permanova was used to statistically compare groups. C, D) Variance between samples was significantly lower in duplicate negative controls compared to urine and stone samples.

FIG. 6. Batch correction. PCoA plot based on a Binomial distance matrix showing differences in the urinary metabolome by batch, before batch correction (A) and after batch correction (B). PERMANOVA was used to statistically compare metabolome composition between groups.

FIG. 7. Metabolomic profile of the kidney stones and urine, stratified by stone composition and recurrence status. Metabolites are only listed if they comprised >0.1% of the metabolome. Metabolites are listed regardless of if they were given putative ID or not. Data indicate that untargeted metabolomic analysis accurately captured the typical urinary metabolome profile.

FIG. 8. Prevalence of metabolites commonly present in urine and calcium-based stones.

### DETAILED DESCRIPTION

The present invention relates to systems, kits, and methods for identifying subjects with increased levels (e.g., in a urine sample) of at least two urine metabolites selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether (or at least two urine metabolites selected from Table 5, Appendix A), as well as methods of determining if a subject has or is at risk of urinary stone disease based on such urine metabolites. In certain embodiments, the urinary tract of a subject with elevated levels of at least two of the urine metabolites is imaged (e.g., to generate an image showing the size, location, or number of urinary stones present).

Urinary stone disease (USD) affects more than 11% of the population with half of those individuals exhibiting at least one recurrent episode. Work conducted during development of embodiments herein observed that threshold urinary concentrations of at least 10 known specific small molecules are useful in predicting an active stone burden. As such, provided herein, in some embodiments, is a chromatography-mass spectrometry based assay, using a spot urine collection, to determine if patients have a recurrent kidney stone assay. Such assays, in certain embodiments, may eliminate repeated imaging protocols based on loose guidelines¹¹, which are burdensome, costly, and exposes patients to unnecessary radiation. Furthermore, spot urine samples could be mailed to the clinic so that patients do even need an appointment for follow-up surveillance.

Current follow-up surveillance for patients at risk of recurrent kidney stone formation often involves regular imaging-based studies or 24-hour urine collections. These measures are expensive, difficult to comply with, and can involve the risk of significant radiation exposure. The proposed solutions provide, in some embodiments, a novel diagnostic test, based on a spot urine sample (or other urine sample) that could be mailed in by the patient. Such a test is easy to comply with, does not require an appointment, and is a tiny fraction of the cost to implement than current measures.

In some embodiments, the control values herein (e.g., for each urine metabolites herein) are derived from samples from the general public or from a group known to not have urinary stone disease or be at risk for urinary stone disease. In certain embodiments, levels of the at least two urine metabolites in the sample obtained from the subject may compared to a control value (e.g., to know if a particular urine metabolite is the same, increased, or decreased compared to the control). A control value is, for example, a concentration of a urine metabolite that represents a known or representative amount of an analyte. For example, the control value can be based upon levels of the selected urine metabolites in comparable samples obtained from a reference cohort. In certain embodiments, the reference cohort is the general population. In certain embodiments, the reference cohort is a select population of human subjects. In certain embodiments, the reference cohort is comprised of individuals who have not previously had any signs or symptoms indicating the presence of urinary stone disease. In certain embodiments, the reference cohort includes individuals, who if examined by a medical professional would be characterized as free of symptoms of disease (e.g., urinary stone disease).

The control value is preferably measured using the same units used to characterize the level of the selected urine metabolite obtained from the subject. Thus, if the level of the selected urine metabolite is an absolute value such as the units of the urine metabolite per ml of blood or plasma, the control value is also based upon the units of the urine metabolite per ml of blood or plasma in individuals in the general population or a select population of human subjects.

The control value can take a variety of forms. The control value can be a single cut-off value, such as a median or mean. The control value can be established based upon comparative groups such as where the risk in one defined group is double the risk in another defined group. The control values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having urinary stone disease can be based upon which group his or her test value falls. Control values of the selected urine metabolite in biological samples obtained, such as mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described, for example, in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each of the urine metabolites that are assayed.

Levels of at least two selected urine metabolites in a subject's biological sample may be compared to a single control value or to a range of control values. In certain embodiments, the at least two urine metabolites are selected from those in Table 5 (Appendix A) or from the following: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether. If the level of the at least two urine metabolites are greater than the control value or exceeds or is in the upper range of control values, the test subject is at greater risk of developing or having urinary stone disease. In certain embodiments, the extent of the difference between the test subject's at least two urine metabolite levels and control values is also useful for characterizing the extent of the risk and thereby determining which individuals would most greatly benefit from certain therapies. In those cases, where the control value ranges are divided into a plurality of groups, such as the control value ranges for individuals at high risk, average risk, and low risk, the comparison involves determining into which group the test subject's level of the relevant risk predictor falls.

Another type of control value is an internal standard in the sample. An internal standard is a known amount of another compound that can be provided in a sample that can be measured along with the analyte to serve as a reference. The diagnostic methods described herein can also be carried out by determining the levels of at least two selected urine metabolites in a subject's biological sample and comparing them to the amount of an internal standard.

### EXAMPLES

### Example 1

To address questions about the lithogenic potential of the urinary metabolome, we sought to: 1) Characterize the non-crystalline metabolome of CBS and urine of SF with or without radiographic stone appearance; and 2) Delineate hypotheses about the influence of urinary metabolites on lithogenesis. While not limiting the present invention, we hypothesize that specific metabolites within the urinary tract may facilitate stone formation through the direct interaction with mineralized components present in the stones.

### Materials and Methods

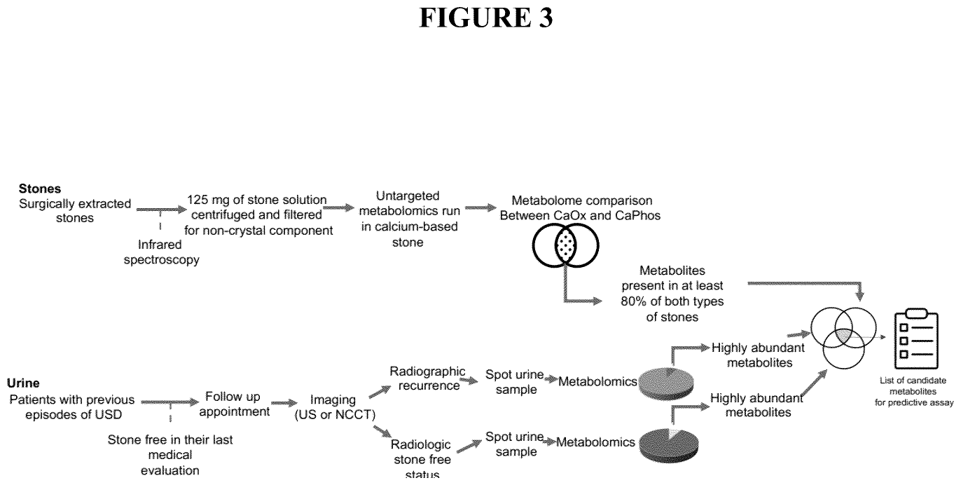

Project workflow is presented in FIG. 3.
Recruitment of participants

### Population to Delineate Metabolome of CBS

To identify stone metabolites, surgically-extracted stones were collected, washed and sent for composition analysis with infrared spectroscopy. Only CBS were considered for metabolomic analysis, which represent about 90% of all USD cases, to focus hypotheses while covering the most commonly manifested stone types³. Pure CaOx or pure CaPhos stones were used to ensure a clear demarcation in stone composition. No other clinical data was collected.

Population to Delineate Urinary Metabolome of Active Vs. Non-Active Stone Formers

Two independent cohorts were recruited for urine specimens to delineate hypotheses surrounding direct and passive metabolite-stone interactions by focusing on patients with a history of USD that visited the Kidney Stone Clinic at Cleveland Clinic to evaluate stone burden by radiographic imaging. These patients either did or did not exhibit radiographic stone activity with imaging. Recruitment of independent populations for the stone and urine analyses allowed for robust hypothesis testing while removing biases associated with individual variabilities in each cohort. The inclusion criteria were patients of both sexes, >18 years old, any ethnicity, history of ≥1 episodes of USD, any composition of previous calculi, stone free after their latest stone episode (no visible stones on imaging) and patients were to be radiologically evaluated to determine stone activity. Patients without imaging on the same day of urine collection and those with an active urinary tract infection were excluded. For imaging, either ultrasonography or non-contrast enhanced computer tomography (NCCT) was used as requested by the treating physician as part of standard operating procedures. Patients were classified as radiographically active, if stones ≥4 mm were observed on imaging, or non-active if no stone was observed. This stone size threshold was used to overcome the sensitivity limitation of ultrasonography¹². Clinical data collected from USD patients were number of previous episodes, method of last episode stone clearance, and time since last USD episode. Given limitations in identifying stone composition based on radiographic imaging, our study is limited to identifying urinary metabolites associated with different types of stones, rather than metabolites specifically linked to CBS. Urine samples were collected and stored based on a established protocol for urine metabolomics research¹³. To minimize diurnal variation and effect of diet on the urinary metabolome, fasting patients were asked to give the first morning, midstream urine samples the same day of clinical follow-up imaging. Urine samples were stored at 4° C., less than 3 h before centrifuging at 14000 RPM for 5 minutes and saving 1 ml of supernatant at −80° C. until processing. All procedures were approved by the Institutional Review Board of Cleveland Clinic (IRB #18-586).

### Untargeted Metabolomics

Urine supernatant and 125 mg of powder-stone samples were diluted 1:4 in a 50% acetonitrile solution containing two internal standards, using previously validated protocols¹⁴. Samples were vortexed to solubilize adherent metabolites and centrifuged at 14,000 g for 5 minutes to precipitate proteins and the supernatant recovered. Supernatant of the stone samples was filtered (0.2 micron) to exclude crystalline components. External standards were added to all samples to ensure run consistency. Negative controls including extraction solutions with added standards were run at the beginning, middle, and end of the run. Untargeted metabolomics was performed on an ultra-high performance liquid chromatography tandem mass spectrometry system coupled to a Q

### Exactive HF Hybrid Quadrupole-Orbitrap Mass Spectrometer.

Untargeted metabolomic data is semi-quantitative and requires normalization to a common factor¹⁴. As such, data from urine samples were normalized to total creatinine quantified through mass spectrometry and data from stones were normalized to total mass used for extraction. Normalized data were analyzed in Metabolyzer software¹⁵. Spectral features were defined by molecular mass and retention time and given putative identification by comparison to metabolites in the curated databases KEGG, HMDB, BioCyc, and LIPIDMAPS¹⁵ with validation of identification given by correctly identifying added internal and external standards from mass and retention time. Prior to comparative analyses, metabolites present in negative controls or in fewer than 25% of samples for any one population were removed. Additionally, samples that had concentrations of internal or external standards that deviated more than 1.5 fold from the inter-quartile range for all samples, were removed.

### Statistical Analysis

Power analysis to calculate the sample size for the cohort of active or non-active patients for urinary metabolome analysis was conducted in the PWR package in R statistical software using results from a previous urinary metabolome study in patients with or without USD⁵.

The CBS samples were stratified into CaOx and CaPhos groups based on composition analysis. Urine samples were stratified into radiographically active vs. non-active. Urine specimens collected for metabolomic analysis were collected, processed and assayed in two batches by the same individuals, one year apart. The urinary metabolomic data was corrected for batch effects with the BER package in R¹⁶, which uses a linear regression model that identifies the location and scale of batch effects in the metabolomic data.

To determine if the metabolomes differed by sample type, stone composition, or radiographic stone activity, the dissimilarity of whole metabolome was quantified through a binomial dissimilarity matrix analysis¹⁷. For stone-urine comparisons, an unweighted analysis, which considers the presence or absence of metabolites, was used given the differences in normalization procedures. For stone-stone or urine-urine comparisons, a weighted analysis, which considers presence or absence of metabolites combined with creatinine-normalized concentration. Dissimilarity between groups was compared by PERMANOVA with 999 permutations, using the Vegan package¹⁸ in R.

To determine the specific metabolites that differentiated each group, a permuted Welch's t-test was conducted. All p-values were corrected via the Benjamini-Hochberg¹⁸ step-up procedure for false discovery rate (FDR) correction. To delineate either the direct interaction of metabolites with the stone matrix vs. passive uptake, the metabolites significantly enriched in the urine of active or non-active SF were compared to metabolites that were highly prevalent in CaOx or CaPhos stones, defined as present in >80% of samples for each stone composition.

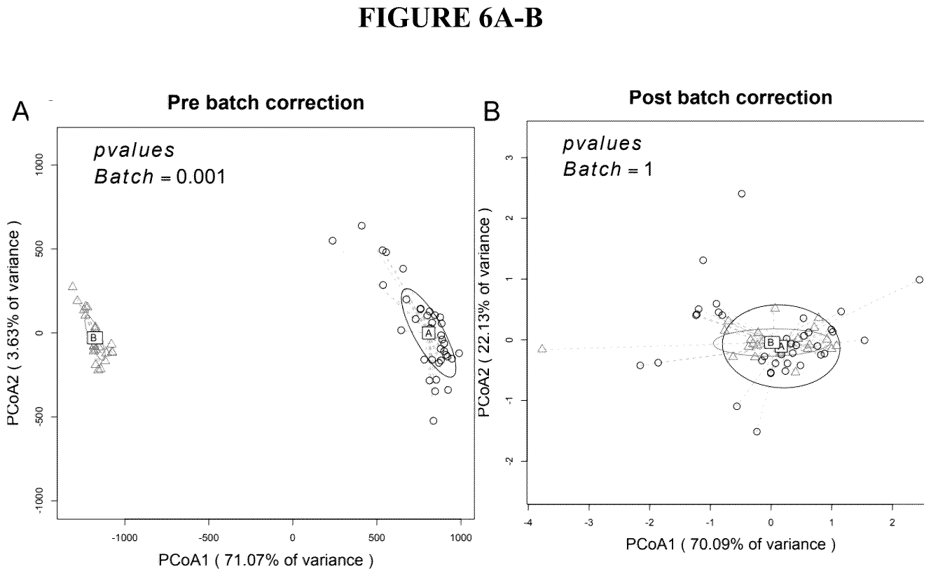

To further validate the relevance of metabolites, we defined threshold concentration values for the metabolites that were both enriched in either the radiographically active or non-active groups and were highly abundant in CaOx or CaPhos stones. Thresholds were quantified by taking the average concentration of the selected metabolites in each group±standard error, then finding the middle value between metabolite concentration in the active population minus standard error and the metabolite concentration in the non-active population plus standard error. Patients with a metabolite concentration above or below the threshold were classified as active or non-active for that metabolite, respectively (FIG. 6). Final classification was based on the majority classifications of the metabolites. These threshold-based predictions were compared to the actual imaging result to determine the error rate between the predicted phenotype and the phenotype based on imaging.

### Results

### Demographics

A total of 10 CaOx stones and 13 CaPhos stones were assessed. Additionally, 60 patients were recruited for urinary metabolome analyses with 40 patients exhibiting an active stone burden and 20 non-active patients. Power analysis for the comparison of the urinary metabolome between active and non-active patients revealed an 86% probability of detecting a significant difference in these populations if one exists. Clinical data of the patients recruited for metabolomics of urine specimens are presented in Table 1.

TABLE 1
Demographic and clinical characteristics of patients providing urine speciemens

| Patient Characteristics | Active SF | | Non-Active SF | | P-Value | Statistic |
|---|---|---|---|---|---|---|
| No. of patients | 40 | | 20 | | NA | NA |
| Age +/− SD | 58 +/− 12 | | 58 +/− 10 | | .22 | Students t test |
| % Male | 50% | | 60% | | .67 | Relative risk, Fisher exact text |
| # of previous stone episodes, median, (IQR) | 1.5 | (1-2) | 1.75 | (1-2) | .513 | Students t test |
| Mean of last episode stone removal | | | | | | |
| Surgically (URS, PNL) | 50% | | 60% | | .217 | Relative risk, Fisher exact text |
| Pass stone | 50% | | 40% | | | |
| Time [symbol] last episode in months, median (IQR) | 22 | ( [symbol] -59) | 24 | (6-142) | .89 | Students t test |
| Imaging [symbol] for actual diag [symbol] | | | | | | |
| % Unenhanced CT scan | 65% | | 20% | | <.001 | Relative risk, Fisher exact text |
| % Ultra- [symbol] graphy | 35% | | 80% | | | |
| Stone characteristics | | | | | | |
| Stone size, median, (IQR) (mm) | 7 | (4-17) | NA | | NA | NA |
| Mean number, (IQR) | 1.4 | (1-3) | NA | | NA | NA |
| Location | | | | | | |

| | | | | |
|---|---|---|---|---|
| % Renal | 87% | NA | NA | NA |
| % Ureteral | 13% | NA | NA | NA |
| % Unilateral | 71% | NA | NA | NA |
| % Bilateral | 29% | NA | NA | NA |

IQR = Interquartile Range. [symbol]
indicates data missing or illegible when filed

Groups did not differ by age, biological sex, method of last stone removal, and time since last active episode, minimizing any biases associated with life history or the natural history of the stones. However, there was a significant difference between groups in the diagnostic imaging modality between groups (Table 1).

### Urine and Stone Metabolome Composition

Normalized values of raw metabolomic data are provided in Table 3.

TABLE 3
Demographic and clinical characteristics of patients providing urine specimens.

| | Radiographic | | No radiographic | | p - | |
|---|---|---|---|---|---|---|
| Patient characteristics | recurrence | | recurrence | | value | Statistic |
| No. of patients | 40 | | 20 | | NA | NA |
| Age +/− SD | 58 +/− 12 | | 58 +/− 10 | | 0.22 | Students t- |
| | | | | | | test |
| % Male | 50% | | 60% | | 0.67 | Relative |
| | | | | | | risk, Fisher |
| | | | | | | exact text |
| # of previous stone | 1.5 | (1-2) | 1.75 | (1-2) | 0.513 | Students t- |
| episodes, median, (IQR) | | | | | | test |
| Mean of last episode | | | | | | |
| stone removal | | | | | | |
| Surgically | 50% | | 60% | | 0.217 | Relative |
| (URS, PNL) | | | | | | risk, Fisher |
| Pass stone | 50% | | 40% | | | exact text |
| Time since last episode in | 22 | (5-59) | 24 | (6-142) | 0.89 | Students t- |
| months, median (IQR) | | | | | | test |
| Imaging modality for | | | | | | |
| actual diagnosis | | | | | | |
| % Unenhanced | 65% | | 20% | | <0.001 | Relative |
| CT scan | | | | | | risk, Fisher |
| % Ultra- | 35% | | 80% | | | exact text |
| sonography | | | | | | |
| Stone characteristics | | | | | | |
| Stone size, median, (IQR) | 7 | (4-17) | NA | | NA | NA |
| (mm) | | | | | | |
| Mean number, (IQR) | 1.4 | (1-3) | NA | | NA | NA |
| Location | | | | | | |

| | | | | |
|---|---|---|---|---|
| % Renal | 87% | NA | NA | NA |
| % Ureteral | 13% | NA | NA | NA |
| % Unilateral | 71% | NA | NA | NA |
| % Bilateral | 29% | NA | NA | NA |

IQR = Interquartile Range

The processed and filtered spectral features with number of metabolites in each population and putative identification are presented in Table 4.

TABLE 4
Total number of processed spectral features in each
population along with the number putatively identified
and without putative identification.

| | 13 CaPhos/ | 26/Active/10 Non- | 14 Active/10 Non- |
|---|---|---|---|
| Study groups | 10 CaOx | Active (BathchA) | Active (BatchB) |
| Total spectral | 5581 | 4409 | 2885 |
| features | | | |
| Features with | 2502 | 2261 | 1673 |
| putative ID | | | |
| Unknowns | 3079 | 2148 | 1212 |

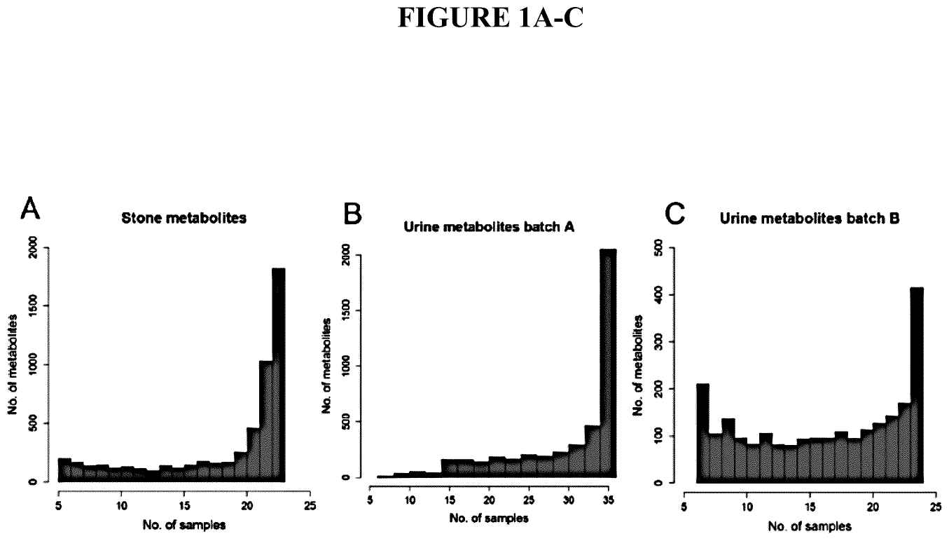

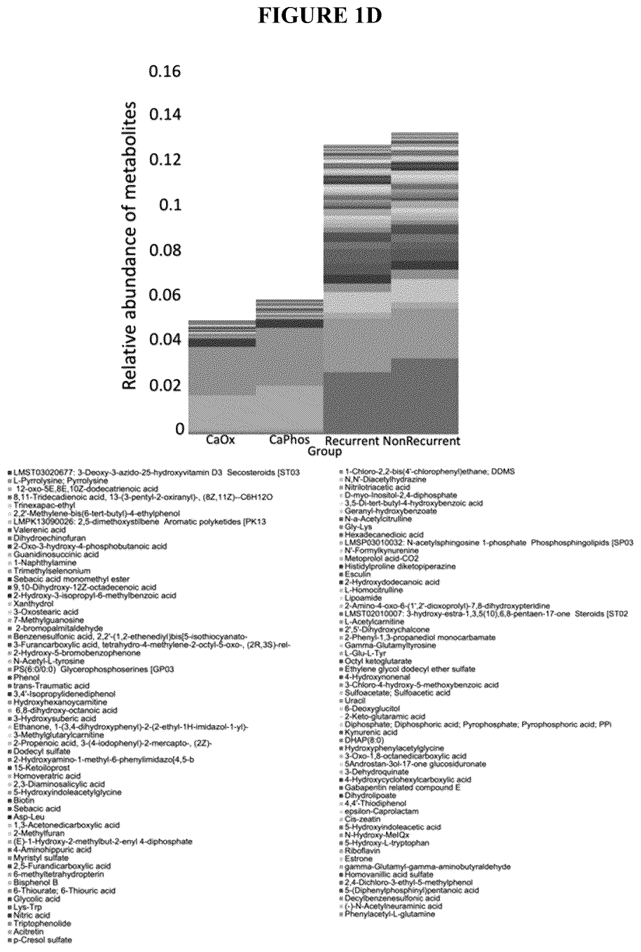

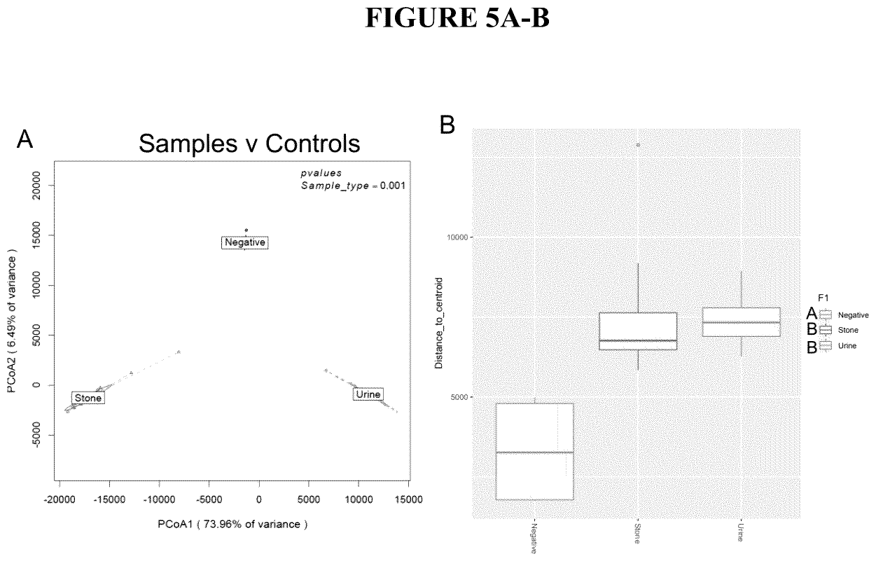

The sample metabolomes differed significantly from negative controls and exhibited significantly greater levels of variance compared to duplicate control samples (FIG. 5). In total, we detected between 2800-4500 urinary metabolites and over 5500 metabolites in the stone matrix (Table 4). The urinary metabolomes, based on specimens collected and processed one year apart, exhibited a significant batch effect (FIG. 6a). However, after batch correction in the BER package, batch effects were effectively removed (FIG. 6b). For both the stone and urinary metabolomes, a core metabolome was apparent, whereby most of the metabolites were present in all or nearly all of the samples assessed (FIG. 1a-b).

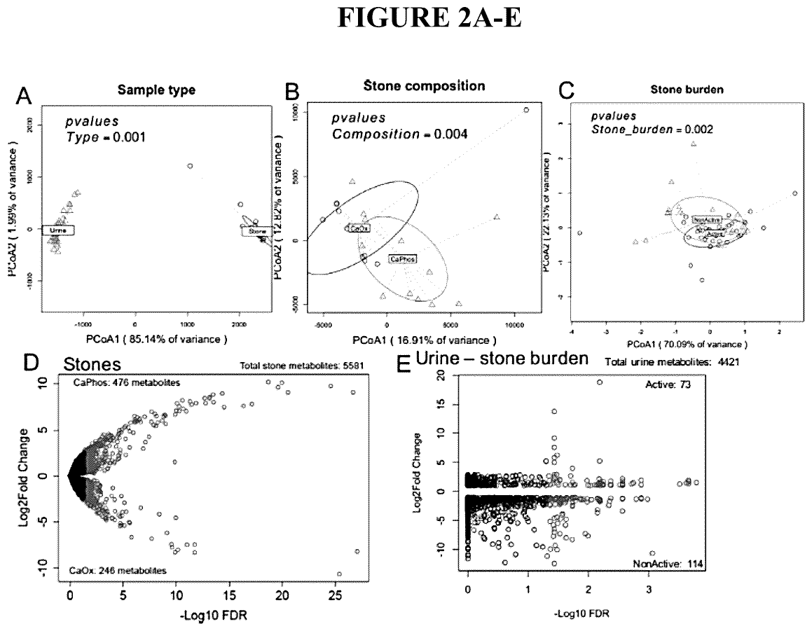

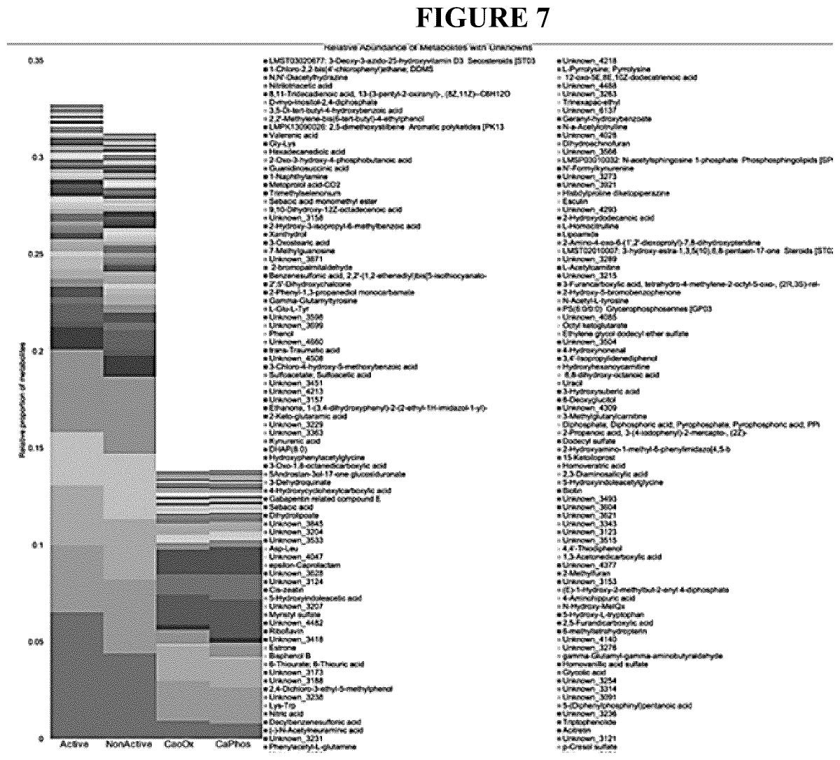

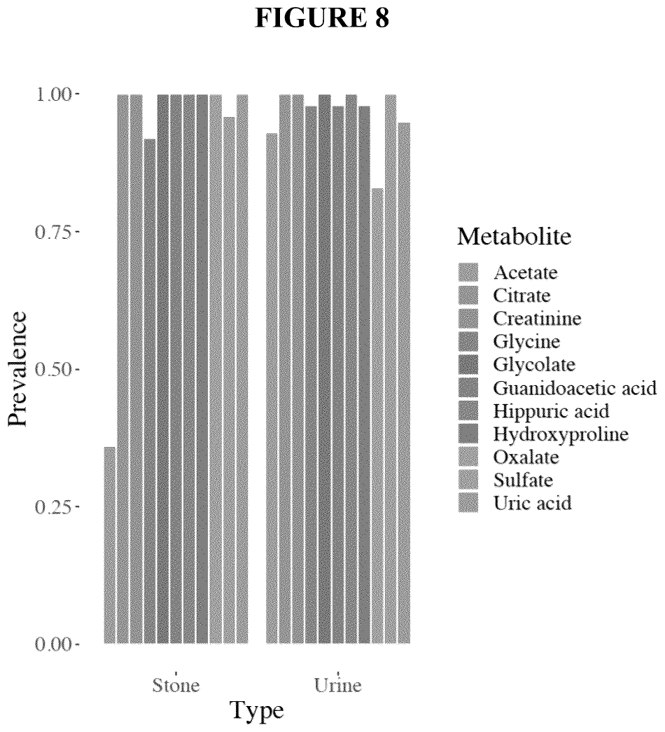

Metabolomic profiles stratified by stone type and radiographic stone activity status are presented in FIGS. 1c and 7. Metabolites commonly present in the urinary metabolome¹⁹, such as oxalate, acetate, uric acid, and creatinine, were almost universally detected in both the urine and stone samples (FIG. 8). Metabolomes differed significantly by sample type (FIG. 2a; Permanova p-value=0.001); stone composition (FIG. 2b; Permanova p-value=0.004); and radiographic stone activity (FIG. 2c; Permanova p-value=0.002).

The differences in the metabolomic composition of the stone types were driven by 722 metabolites enriched in either the CaOx or CaPhos stones (FIG. 2d). Differences in the urinary metabolome between active vs non-active SF were driven by 187 metabolites enriched in either group (FIG. 2e). The complete list of significantly different metabolites between groups is given in Table 5.

Table 5. Metabolites that were significantly different between groups. Listed are the dataset compared (initial or validation), group the metabolite was enriched in (i.e. CaOx, CaPhos). Table 5 is at Appendix A.

### Interactions Between Urinary and Stone Metabolomes

To help delineate potential interactions between the urinary metabolome and stone burden, we cross-referenced metabolites significantly enriched in the urine of active and non-active SF to those that were highly prevalent in CaOx or CaPhos stones, defined as being present in >80% of stone samples. The 80% threshold was selected to focus only on metabolites strongly associated with the stone matrix. This approach revealed that of the 73 metabolites enriched in active SF, 27 were highly prevalent in CaOx stones and 14 in CaPhos stones. All 14 highly prevalent metabolites in CaPhos stones were also highly prevalent in CaOx stones (Table 2).

TABLE 2
Interaction between stone burden and stone metabolome

| Group | Total No. of [symbol] | C [symbol] ( [symbol] ) | C [symbol] ( [symbol] ) | [symbol] C [symbol] |
|---|---|---|---|---|
| Active | 73 | 27 | [symbol] | [symbol] |
| Non-Active | 1 [symbol] 4 | 0 | 0 | 0 |
| Mass [symbol] | [symbol] | [symbol] ID | | |
| 73. [symbol] | [symbol] 4.33 | [symbol] | | |
| [symbol] . [symbol] | [symbol] 7.37 | Unknown | | |
| 10 [symbol] | [symbol] 7.28 | Unknown | | |
| −3 [symbol] .05 | 1.8 [symbol] | [symbol] | | |
| −20 [symbol] 2 | [symbol] 0.67 | 6-M [symbol] | | |
| 20 [symbol] | 12.8 | [symbol] | | |
| −1 [symbol] | [symbol] 0.64 | [symbol] | | |
| −23 [symbol] | [symbol] 0.64 | Unknown | | |
| − [symbol] 72 [symbol] | [symbol] | [symbol] | | |
| −2 [symbol] | [symbol] .91 | Unknown | | |
| − [symbol] | 2.3 [symbol] | Methyl [symbol] | | |
| 19 [symbol] 7 | [symbol] 1.65 | [symbol] | | |
| − [symbol] 7 [symbol] . [symbol] 7 | [symbol] 0.48 | 2-O [symbol] | | |
| −3 [symbol] | 8.5 [symbol] | 2-Hyd [symbol] ether | | |
| | [symbol] ( [symbol] ) | [symbol] ( [symbol] ) | [symbol] | T [symbol] |
| A [symbol] ( [symbol] ) | 24 | 16 | 4 [symbol] % | 26. [symbol] % |
| Non-A [symbol] ( [symbol] ) | 0 | 20 | 0 | |

(Top) Number of [symbol] [symbol]
indicates data missing or illegible when filed

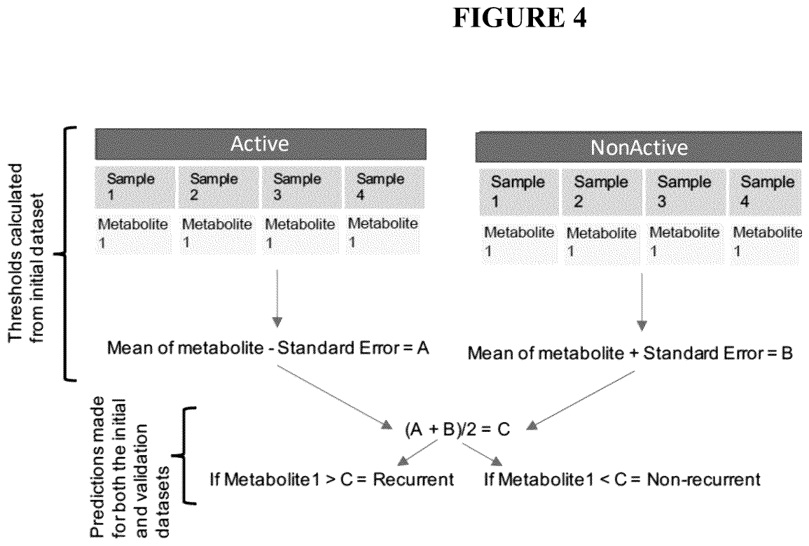

In contrast, none of the metabolites enriched in the non-active SF were found to be highly prevalent in either CaOx or CaPhos stones (Table 2). Ten of the 14 metabolites that were significantly enriched in the urine of active SF and highly prevalent in CBS were putatively identified (Table 2). To further validate that these specific metabolites could be drivers of stone activity, we developed a predictive radiographic activity assay after establishing threshold values for metabolite concentrations using data from the initial cohort (FIG. 4). With this assay, we predicted radiographic stone activity with 73.3% success rate (Table 2). All of the error in the prediction was generated by falsely classifying active SF as non-active SF.

Case-control studies have revealed significant differences in the whole metabolome between individuals with an active episode of USD and those with no history of the disease^{5, 6}. The current study sought to delineate hypotheses about urinary metabolites present in the stone matrix, as potential promoters of lithogenesis or as passive components absorbed into organic matrix of the stone. Several metabolomic characteristics distinguish the alternative hypotheses. Under the direct promoter hypothesis, we would expect unique metabolomes between CaOx and CaPhos stones. In contrast, under the passive absorption hypothesis, we would expect no such metabolomic differences. Here we show clear differences in the metabolome of CaOx vs. CaPhos stones, which supports the direct promoter hypothesis (FIG. 2b), although the present invention is not limited to any particular mechanism or hypothesis in order to practice the invention. Interestingly, there were clear differences in the metabolites present in stones of both types and the urine (FIG. 2a). These data may reflect the fact that the urinary metabolome is transient, influenced by day-to-day activities⁷ whereas kidney stones are a longer-term record of bioactivity²⁰. Importantly, our urine specimens come from fasting patients, eliminating much of the variability¹³.

The second characteristic that distinguishes the direct promoter and passive absorption hypotheses refers to differences in the urinary metabolome of individuals with a history of USD, with or without radiographic stone activity. We observed a significant difference in the urinary metabolome profile based on the radiographic presence of a stone (FIG. 2c). Taken alone, these data suggest that having a stone may impede metabolite excretion through passive absorption into the stone matrix, as a result of differential binding affinities between metabolites and components of the stone matrix. However, these data cannot rule out the hypothesis that individuals with radiographically active stones have higher concentrations of metabolites that do actively promote lithogenesis, necessitating further analysis.

To validate the passive uptake hypothesis, we compared the metabolites significantly enriched in the urine of active vs. non-active SF to those that were highly prevalent in the CBS. With this analysis, under the stone promoter hypothesis, we expect to see an overlap of metabolites enriched in the urine of patients with radiographic activity and those that are highly prevalent in the stones, with the assumption that higher levels of stone-promoting urinary metabolites would facilitate urinary stone development. In contrast, with passive absorption, we would expect that stones will become part of the stone matrix without promoting lithogenesis. This would produce the counter-intuitive result that there would be an overlap in metabolites enriched in non-active SF and those that are highly prevalent in stones. Comparison of metabolites differing between active and non-active SF clearly favored the stone-promoter hypothesis (Table 2), such that there was an overlap of several of the metabolites present in the group with stone activity and the metabolites highly prevalent in stones, but there was no overlap between the metabolites in the non-active group and the metabolites prevalent in stones. These results are in contrast to the interpretation achieved using the urine metabolome data alone, but consistent with the comparative stone metabolome data. Collectively, results suggest that the urine of active SF has a higher concentration of potentially lithogenic metabolites than non-active SF.

Among the potentially lithogenic metabolites identified, ten were given putative identification. Of these, butanal²¹, N-butanoyl-lhomoserine lactone²², and methyl-propenyl-ketone²³ have been correlated with pathogenic bacteria activity, supporting a potential role of the urinary microbiome in stone formation⁷. Six metabolites were of apparent host origins. These include the metabolite 6-methylmercaptopurine, which has previously been associated with lithogenic activity²⁴. Hexanolglycine has been closely linked with metabolic syndrome²⁵. Two of the metabolites are involved in estrogen metabolism pathways, 2-hydroxyestradiol-3-methyl ether, and methylenedioxycoumestan²⁶. Previous work has demonstrated that renal cells treated with estrogen exhibit reduced CaOx crystal binding which may contribute to stone prevention²⁷, so weaker estrogenic activity represented by these metabolites could be associated with a diminished protective effect of estrogen. Two additional metabolites were derivatives of arginine, dimethyl-L-arginine and 2-oxoarginine. Dimethyl-L-arginine is a biomarker of proteolysis²⁸ and high levels are associated with chronic kidney disease, end-stage renal disease, and coronary calcifications²⁹. The metabolite 2-oxoarginine is formed during the catabolism of arginine and has been linked to sepsis in rodent models³⁰. Ferulate is ubiquitously produced in plants and likely represents some residuals from the diet³¹. Future studies will need to address the causality of the metabolites identified here for stone formation.

The metabolome of CaOx vs. CaPhos stones differs significantly, as does the urinary metabolome of radiologically active and non-active SF. Collectively, our data suggest that stone activity may be driven by high levels of lithogenic metabolites in the urinary tract that can potentially promote lithogenesis.

### REFERENCES

1. Pearle et al.: Medical management of kidney stones: AUA guideline. The Journal of urology, 192: 316-324, 2014
2. Litwin, M., Saigal, C.: Economic impact of urologic disease. Urologic diseases in America, 494, 2012
3. Scales et al.: Urinary stone disease: advancing knowledge, patient care, and population health. Clinical Journal of the American Society of Nephrology, 11: 1305-1312, 2016
4. Worcester, E. M., Coe, F. L.: Calcium kidney stones. New England Journal of Medicine, 363: 954-963, 2010
5. Zampini et al.: Defining Dysbiosis in Patients with Urolithiasis. Scientific reports, 9: 1-13, 2019
6. Duan et al.: 1H NMR-based metabolomic study of metabolic profiling for the urine of kidney stone patients. Urolithiasis, 48: 27-35, 2020
7. Khamis, M. M., Adamko, D. J., El-Aneed, A.: Mass spectrometric based approaches in urine metabolomics and biomarker discovery. Mass spectrometry reviews, 36: 115-134, 2017
8. Manzoor et al.: Morphological and micro-tomographic study on evolution of struvite in synthetic urine infected with bacteria and investigation of its pathological biomineralization. PloS one, 13: 1-12, 2018
9. Torricelli, et al., Urolithiasis in complicated inflammatory bowel disease: a comprehensive analysis of urine profile and stone composition. International Urology and Nephrology, 53: 205-209, 2021
10. Anan et al.: The impact of glycosylation of osteopontin on urinary stone formation. International journal of molecular sciences, 21: 93, 2020
11. Brisbane, W., Bailey, M. R., Sorensen, M. D.: An overview of kidney stone imaging techniques. Nature Reviews Urology, 13: 654-662, 2016
12. Bezabeh, T., Capati, A., Ijare, O. B.: NMR-Based Urinary Metabolomics Applications. In: NMR-Based Metabolomics: Springer, pp. 215-229, 2019
13. Goudarzi et al.: Development of urinary biomarkers for internal exposure by cesium-137 using a metabolomics approach in mice. Radiation research, 181: 54-64, 2013
14. Mak et al.: Metabolyzer: A novel statistical workflow for analyzing postprocessed lc-ms metabolomics data. Analytical chemistry, 86: 506-513, 2013
15. Giordan, M.: A two-stage procedure for the removal of batch effects in microarray studies. Statistics in Biosciences, 6: 73-84, 2014
16. Qi, Z., Voit, E. O.: Strategies for comparing metabolic profiles: Implications for the inference of biochemical mechanisms from metabolomics data. IEEE/ACM transactions on computational biology and bioinformatics, 14: 1434-1445, 2016
17. Oksanen et al.: Vegan: community ecology package. R package version 1.17-4. <http://cran.r-project.org>. Acesso em, 23: 2010, 2010
18. Takis et al.: Deconvoluting interrelationships between concentrations and chemical shifts in urine provides a powerful analysis tool. Nature communications, 8: 1-12, 2017
19. Sivaguru et al.: Geobiology reveals how human kidney stones dissolve in vivo. Scientific reports, 8: 1-9, 2018
20. Timm et al.: Direct growth of bacteria in headspace vials allows for screening of volatiles by gas chromatography mass spectrometry. Frontiers in microbiology, 9: 491, 2018
21. Alayande, A. B., Aung, M. M., Kim, I. S.: Correlation between quorum sensing signal molecules and Pseudomonas aeruginosa's biofilm development and virulency. Current microbiology, 75: 787-793, 2018
22. Garner, C. E., Smith, S., de Lacy Costello, B. et al.: Volatile organic compounds from feces and their potential for diagnosis of gastrointestinal disease. The FASEB Journal, 21: 1675-1688, 2007
23. Shadman, A., Bastani, B.: Evaluation and Management of Kidney Calculi. Iranian journal of kidney diseases, 11: 395, 2017
24. Cho et al.: Correlation of metabolic syndrome with urinary stone composition. International Journal of Urology, 20: 208-213, 2013
25. Jackson, C.-J. C., Rupasinghe, H. V.: Human Dietary Sources of Phytoestrogens and. Phytoestrogens and Health: 95, 2002
26. Peerapen, P., Thongboonkerd, V.: Protective Cellular Mechanism of Estrogen Against Kidney Stone Formation: A Proteomics Approach and Functional Validation. Proteomics, 19: 1-15, 2019
27. Tsikas, D.: Urinary dimethylamine (DMA) and its precursor asymmetric dimethylarginine (ADMA) in clinical medicine, in the context of nitric oxide (NO) and beyond.

Journal of clinical medicine, 9: 1843-1862, 2020
28. Mallamaci, F., Zoccali, C.: Clinical implications of elevated asymmetric dimethylarginine in chronic kidney disease and end-stage renal disease. Journal of Renal Nutrition, 19: 25-28, 2009
29. Lin et al.: Comparison of sepsis rats induced by caecal ligation puncture or Staphylococcus aureus using a LC-QTOF-MS metabolomics approach. Infection, Genetics and Evolution, 43: 86-93, 2016
30. Mota, et al.: Aqueous solubility of some natural phenolic compounds. Industrial & Engineering Chemistry Research, 47: 5182-5189, 2008.

All publications and patents mentioned in the present application are herein incorporated by reference. Various modification and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

TABLE 5
APPENDIX A

| Sample | | | Molecular- | | log2Fold | |
|---|---|---|---|---|---|---|
| type | Group | Putative Metabolite ID | mass | ESI | Change | FDR |
| Urine | Active | Tetrahydrocortisone | 365.23 | Positive | −8.2560217 | 0.01867989 |
| Urine | Active | 2,5-Dichloro-cis,cis-muconate | 244.94 | Negative | −5.1732558 | 0.04386822 |
| Urine | Active | 2-Hydroxyestradiol-3-methyl | 301.18 | Negative | −4.0405031 | 0.02735055 |
| | | ether | | | | |
| Urine | Active | Palmitaldehyde | 263.24 | Positive | −3.7949413 | 0.02712935 |
| Urine | Active | N-Acetyl-L-phenylalanine | 208.1 | Positive | −2.3611931 | 0.01854201 |
| Urine | Active | 1/1,[7-Aminomethyl-7- | 178.07 | Negative | −2.0674362 | 0.02918261 |
| | | carbaguanine] | | | | |
| Urine | Active | butyl 3S-hydroxybutanoate | 455.21 | Positive | −1.9625883 | 0.02466182 |
| Urine | Active | 2-Hydroxy-1,3-dimethoxy-8,9- | 355.05 | Negative | −1.9409819 | 0.01854201 |
| | | methylenedioxycoumestan | | | | |
| Urine | Active | 2,[CPD0-1143: azaguanine], | 175.03 | Positive | −1.7195237 | 0.02445409 |
| | | [CYSTAMINE: cystamine] | | | | |
| Urine | Active | Dopamine glucuronide | 330.12 | Positive | −1.6740592 | 0.00447313 |
| Urine | Active | 3,5-Tetradecadiencarnitine | 368.28 | Positive | −1.4086972 | 0.01303066 |
| Urine | Active | 4,7,10-hexadecatrienoic acid | 273.18 | Positive | −1.3778001 | 0.01374541 |
| Urine | Active | Methylarsonate | 141 | Positive | −1.2925199 | 0.01061191 |
| Urine | Active | 2-Oxoarginine | 172.1 | Negative | −1.2761174 | 0.02593255 |
| Urine | Active | 6-Methylmercaptopurine | 201.02 | Negative | −1.2692475 | 0.02918261 |
| Urine | Active | Ferulate | 195.1 | Positive | −1.1517077 | 0.01374541 |
| Urine | Active | L-beta-aspartyl-L-threonine | 235.09 | Positive | −1.0337317 | 0.01886535 |
| Urine | Active | trans-Hex-2-enoic acid | 132.1 | Positive | −1.0140526 | 0.01854201 |
| Urine | Active | Isoniazid | 138.07 | Positive | −1.0268528 | 0.00932697 |
| Urine | Active | Pymetrozin | 218.1 | Positive | −1.0432986 | 0.00275656 |
| Urine | Active | Ofloxacin | 362.15 | Positive | −1.0588221 | 0.00275656 |
| Urine | Active | Vanillylmandelic acid | 197.05 | Negative | −1.0747054 | 0.00591127 |
| Urine | Active | Arg-Gly-Asp | 347.17 | Positive | −1.0811808 | 0.00717055 |
| Urine | Active | LMPK12020121: Epigallocatechin | −457.07 | Negative | −1.1200414 | 0.00275656 |
| | | 3-O-(3-O-methylgallate) | | | | |
| | | Flavonoids [PK12 | | | | |
| Urine | Active | 1-Octadecanoyl-sn-glycero-3- | −511.3 | Negative | −1.1460705 | 0.00302795 |
| | | phospho-(1′-sn-glycerol) | | | | |
| Urine | Active | Phosphoric acid | −96.97 | Negative | −1.1481408 | 0.00790184 |
| Urine | Active | 1,2-Bis(O-octanoyl)-sn- | 532.29 | Positive | −1.1508142 | 0.00134268 |
| | | glycerylphophorylcholine | | | | |
| Urine | Active | Threonic acid | −135.03 | Negative | −1.1579007 | 0.04879527 |
| Urine | Active | Suxibuzone | 461.18 | Positive | −1.1674618 | 0.00134268 |
| Urine | Active | Eriosemaone C | −433.12 | Negative | −1.1699851 | 0.00275656 |
| Urine | Active | N-butanoyl-lhomoserine lactone | 170.08 | Negative | −1.1740572 | 0.02445409 |
| Urine | Active | Rutinose | −307.11 | Negative | −1.1789058 | 0.00684318 |
| Urine | Active | Engeletin | −433.12 | Negative | −1.1816627 | 0.00275656 |
| Urine | Active | Gossypetin 3-methylether | −331.05 | Negative | −1.2834126 | 0.00275656 |
| Urine | Active | 2-Oxo-10-methylthiodecanoic acid | 255.1 | Positive | −1.2910781 | 0.00585929 |
| Urine | Active | Piperazine-N,N′-bis(2- | 385.08 | Positive | −1.3019397 | 0.0019405 |
| | | hydroxypropanesulfonic acid) | | | | |
| Urine | Active | 1-Methyladenosine | 282.12 | Positive | −1.3670081 | 0.00684318 |
| Urine | Active | 15R-PGE2 methyl ester, 15-acetate | 409.26 | Positive | −1.4757543 | 0.00181507 |
| Urine | Active | 1-Heptene | 116.14 | Positive | −1.5425931 | 0.00016374 |
| Urine | Active | Dimethyl-L-arginine | 203.2 | Positive | −1.572937 | 0.01854201 |
| Urine | Active | Hexanoylglycine | 172.1 | Negative | −1.7790808 | 0.00427878 |
| Urine | Active | 1/1,[Oxalacetic acid] | 167 | Negative | −1.8966501 | 0.04441385 |
| Urine | Active | Aminocyclopyrachlor | 214.03 | Positive | −1.8975463 | 0.03007526 |
| Urine | Active | Butanal | 73.07 | Positive | −1.9351451 | 0.00022052 |
| Urine | Active | Phe-Tyr | 329.15 | Positive | −2.0682017 | 0.03007526 |
| Urine | Active | S-[2-(N7-Guanyl)ethyl | −339.09 | Negative | −2.1094855 | 0.00668192 |
| Urine | Active | Baicalin | 447.09 | Positive | −2.1815408 | 0.00668192 |
| Urine | Active | Telmisartan | −513.24 | Negative | −2.2216535 | 0.00275656 |
| Urine | Active | (+)-trans-Chrysanthemic acid | 169.13 | Positive | −2.6443649 | 0.04597113 |
| Urine | Active | Methyl propenyl ketone | 83.05 | Negative | −2.6550523 | 0.01994272 |
| Urine | Active | 4-Pyridoxic acid | 184.06 | Positive | −3.9789901 | 0.01851618 |
| Urine | Active | Wogonoside | 461.11 | Positive | −5.2247771 | 0.00656266 |
| Stone | CaOx | 3-Sulfinoalanine | 186.99 | Negative | −10.656093 | 4.15E−26 |
| Stone | CaOx | Difenacoum | 443.16 | Negative | −8.2037506 | 8.57E−28 |
| Stone | CaOx | PE(P-16:0e/0:0) | 472.28 | Negative | −8.0052477 | 6.79E−11 |
| Stone | CaOx | Tetranor-PGEM-d6 | 331.17 | Negative | −7.736675 | 2.51E−10 |
| Stone | CaOx | C07207 Zalcitabine | 234.09 | Positive | −7.4560002 | 9.47E−12 |
| Stone | CaOx | 1H-Indole-1-pentanoic acid, 3-[2- | 368.11 | Negative | −7.4415949 | 1.74E−12 |
| | | (2-chlorophenyl)acetyl | | | | |
| Stone | CaOx | Farnesylcysteine | 326.22 | Positive | −6.7997098 | 1.99E−08 |
| Stone | CaOx | dihydrophaseic acid 4-O-beta-D- | 462.23 | Positive | −6.1718868 | 7.36E−10 |
| | | glucoside Isoprenoids | | | | |
| Stone | CaOx | C03409 N-Formimino-L-aspartate | 161.06 | Positive | −5.431824 | 1.96E−05 |
| Stone | CaOx | Ibuprofen D-glucuronide | 381.16 | Negative | −5.0968735 | 2.40E−06 |
| Stone | CaOx | His-Ala | 225.1 | Negative | −4.624812 | 0.0004972 |
| Stone | CaOx | Rhapontigenin | 257.08 | Negative | −4.6239887 | 0.00011733 |
| Stone | CaOx | Flufenamic acid | 280.06 | Negative | −4.5414343 | 0.00010495 |
| Stone | CaOx | punaglandin 7 | 439.19 | Positive | −4.4965155 | 0.00043651 |
| Stone | CaOx | Cytidine 2′,3′-cyclic phosphate | 306.05 | Positive | −4.4232642 | 1.95E−05 |
| Stone | CaOx | Fluvastatin | 446.18 | Negative | −4.338913 | 1.28E−05 |
| Stone | CaOx | thyronamine | 264.1 | Negative | −4.2706399 | 0.00023088 |
| Stone | CaOx | Amaronol A Flavonoids | 319.05 | Negative | −4.1353749 | 0.00036128 |
| Stone | CaOx | 1-Benzyl-3-methylpiperidin-4-one | 204.13 | Positive | −4.1305013 | 3.86E−09 |
| Stone | CaOx | Sphingosine 1-phosphate | 414.24 | Negative | −4.0425733 | 0.00199652 |
| Stone | CaOx | S-(2-Hydroxyethyl)glutathione | 386.11 | Negative | −3.9020275 | 1.18E−05 |
| Stone | CaOx | 3,7-Dihydroxy-4,5-dimethoxy-8- | 566.26 | Positive | −3.8768763 | 5.06E−06 |
| | | prenylflavan 7-O-beta-D- | | | | |
| | | glucopyranoside | | | | |
| Stone | CaOx | C05513 Urate-3-ribonucleoside | 301.08 | Positive | −3.8509212 | 0.00077705 |
| Stone | CaOx | 2,3,4′,5-Tetrahydroxystilbene 2- | 405.12 | Negative | −3.8487171 | 0.00029588 |
| | | glucoside | | | | |
| Stone | CaOx | Geranyl pyrophosphate | 295.05 | Negative | −3.8236756 | 5.83E−05 |
| Stone | CaOx | Peridinin | 648.39 | Positive | −3.8138374 | 0.00044084 |
| Stone | CaOx | 25-hydroxy-cholesterol(d3) Sterols | 440.36 | Negative | −3.7175626 | 0.00082912 |
| Stone | CaOx | 3-Methylthiopropyl- | 326.07 | Negative | −3.7002712 | 0.00096571 |
| | | desulfoglucosinolate | | | | |
| Stone | CaOx | ammeline | 126.04 | Negative | −3.6689236 | 0.00213526 |
| Stone | CaOx | cotinine-glucuronide | 352.13 | Negative | −3.5902049 | 0.00143783 |
| Stone | CaOx | Urea-1-carboxylate; Allophanate; | 139.01 | Positive | −3.5547976 | 0.00179551 |
| | | Allophanic acid | | | | |
| Stone | CaOx | Citalopram aldehyde | 330.09 | Negative | −3.4991804 | 0.00042915 |
| Stone | CaOx | 16-bromo-9E-hexadecenoic acid | 333.14 | Positive | −3.4824226 | 0.00530267 |
| Stone | CaOx | Quinaprilat | 409.18 | Negative | −3.2949027 | 0.00305927 |
| Stone | CaOx | Phenytoin | 251.08 | Negative | −3.2832587 | 0.00276868 |
| Stone | CaOx | 1,2,3,4-Tetrahydroacridin-9-ol | 200.11 | Positive | −3.2459362 | 0.00964782 |
| Stone | CaOx | C03077 4-Chlorophenylacetate | 193 | Positive | −3.238166 | 0.00164892 |
| Stone | CaOx | 3-Sulfopyruvic acid | 186.01 | Positive | −3.2267761 | 0.01339937 |
| Stone | CaOx | (S)-3-Hydroxy-N-methylcoclaurine | 333.18 | Positive | −3.2001285 | 0.00466268 |
| Stone | CaOx | 11beta,20-Dihydroxy-3-oxopregn- | 397.2 | Positive | −3.187279 | 0.01097797 |
| | | 4-en-21-oic acid | | | | |
| Stone | CaOx | Deoxyadenosine monophosphate | 330.06 | Negative | −3.184602 | 0.00138386 |
| Stone | CaOx | 17,18-dehydro-clavulone I | 445.22 | Positive | −3.0312727 | 0.00114672 |
| Stone | CaOx | Scopolamine | 302.14 | Negative | −3.0223761 | 0.00472498 |
| Stone | CaOx | 2-(1,3-Benzodioxol-5-yl)-3,5,6,8- | 505.15 | Negative | −3.0195109 | 0.00756544 |
| | | tetramethoxy-7-[(3-methyl-2- | | | | |
| | | butenyl)oxy | | | | |
| Stone | CaOx | 1alpha,25-dihydroxy- | 524.26 | Positive | −3.0050762 | 0.02428016 |
| | | 26,26,26,27,27,27-hexafluoro- | | | | |
| | | 16,17,23,23,24,24-hexadehydro- | | | | |
| | | 19-norvitamin D3/1alpha,25- | | | | |
| | | dihydroxy-26,26,26,27,27,27- | | | | |
| | | hexafluoro-16,17,23,23,24,24- | | | | |
| | | hexadehydro-19- | | | | |
| | | norcholecalciferol Secosteroids | | | | |
| | | [ST03 | | | | |
| Stone | CaOx | Estrone sulfate | 373.11 | Positive | −2.9999279 | 0.02270107 |
| Stone | CaOx | Kurilensoside G | 621.36 | Positive | −2.9676731 | 0.00415181 |
| Stone | CaOx | 3-oxo-octanoyl-[acp | 208.06 | Negative | −2.948957 | 0.00804549 |
| Stone | CaOx | de-Hypoxanthine futalosine; | 295.08 | Positive | −2.9474967 | 0.01786496 |
| | | Dehypoxanthine futalosine | | | | |
| Stone | CaOx | PE(80/00) | 490.25 | Positive | −2.8982006 | 0.00752703 |
| Stone | CaOx | Nicotinuric acid | 172.03 | Negative | −2.8698623 | 0.00048817 |
| Stone | CaOx | 5,7-Dimethoxy-8-prenylflavan | 339.2 | Positive | −2.8124931 | 0.00614768 |
| Stone | CaOx | 16-bromo-9E-hexadecenoic acid | 333.14 | Positive | −2.7937576 | 0.00302155 |
| Stone | CaOx | L-Histidine | 154.06 | Negative | −2.7833444 | 0.00961316 |
| Stone | CaOx | adenosine 5′-phosphoramidate | 345.07 | Negative | −2.7491694 | 0.01650962 |
| Stone | CaOx | 5beta-cholestane | 395.37 | Positive | −2.7312651 | 0.01207561 |
| Stone | CaOx | C16562 Glutathionylspermine | 514.28 | Positive | −2.7218783 | 0.01102482 |
| Stone | CaOx | Ribose 1,5-bisphosphate | 344.98 | Negative | −2.7108973 | 0.01067826 |
| Stone | CaOx | C06868 Carbamazepine | 259.09 | Positive | −2.6867873 | 0.04683293 |
| Stone | CaOx | (3S)-7-hydroxy-2′,3′,4′,5′,8- | 377.16 | Positive | −2.6857763 | 0.02502028 |
| | | pentamethoxyisoflavan | | | | |
| Stone | CaOx | 3-iodo-octadecanoic acid | 433.16 | Positive | −2.6447229 | 0.03253131 |
| Stone | CaOx | 4′-Methoxychalcone Flavonoids | 239.11 | Positive | −2.6427296 | 0.02975471 |
| Stone | CaOx | 2,3-Dihydroxycarbamazepine | 267.08 | Negative | −2.6004804 | 0.00397387 |
| Stone | CaOx | Ophthalmic acid | 324.12 | Negative | −2.5696681 | 6.50E−05 |
| Stone | CaOx | L-2-Aminoadipate adenylate | 489.11 | Negative | −2.5531928 | 0.0263087 |
| Stone | CaOx | C01596 Maleamate | 116.04 | Positive | −2.5260512 | 0.00038114 |
| Stone | CaOx | Geranylfarnesyl diphosphate | 536.29 | Positive | −2.521106 | 0.01774501 |
| Stone | CaOx | 1,3-Dichloropropene | 144.96 | Negative | −2.5061193 | 0.00267449 |
| Stone | CaOx | Indoleacetyl glutamine | 338.11 | Negative | −2.5044392 | 0.02731551 |
| Stone | CaOx | Phosphoguanidinoacetate | 196.01 | Negative | −2.4887636 | 0.01605829 |
| Stone | CaOx | Se-Adenosylselenohomocysteine | 431.06 | Negative | −2.4835053 | 0.04223131 |
| Stone | CaOx | 5-O-(1-Carboxyvinyl)-3- | 323.02 | Negative | −2.4801829 | 0.01569232 |
| | | phosphoshikimate; O5-(1- | | | | |
| | | Carboxyvinyl)-3-phosphoshikimate | | | | |
| Stone | CaOx | N-(tert-Butoxycarbonyl)-L- | 264.13 | Negative | −2.4734686 | 0.04001384 |
| | | phenylalanine | | | | |
| Stone | CaOx | C20386 Biotin sulfoxide | 261.09 | Positive | −2.4692512 | 0.00669761 |
| Stone | CaOx | i > N/i > -acetyl-5- | 271.11 | Negative | −2.3859339 | 0.02854431 |
| | | methoxykynurenamine | | | | |
| Stone | CaOx | Orotidine | 287.05 | Negative | −2.3689833 | 0.00573357 |
| Stone | CaOx | Lithocholyltaurine | 482.29 | Negative | −2.3581656 | 0.0486598 |
| Stone | CaOx | Cerivastatin | 494.24 | Negative | −2.332791 | 0.04098987 |
| Stone | CaOx | 5-(3-Indolylmethylene)barbituric | 254.06 | Negative | −2.3314576 | 0.03344965 |
| | | acid | | | | |
| Stone | CaOx | Urate-3-ribonucleoside; Uric acid | 299.06 | Negative | −2.3240408 | 0.00266712 |
| | | ribonucleoside | | | | |
| Stone | CaOx | Xanthosine | 319.07 | Negative | −2.3140163 | 0.0074127 |
| Stone | CaOx | S-(Indolylmethylthiohydroximoyl)- | 328.07 | Negative | −2.3013162 | 0.0240546 |
| | | L-cysteine | | | | |
| Stone | CaOx | (−)-1,2:5,6-Di-O-cyclohexylidene-L- | 339.18 | Negative | −2.2909813 | 0.03101585 |
| | | inositol | | | | |
| Stone | CaOx | 2-Acetoxyamino-1-methyl-6- | 281.11 | Negative | −2.28475 | 0.01616825 |
| | | phenylimidazo[4,5-b | | | | |
| Stone | CaOx | Kinetin | 250.07 | Negative | −2.2031008 | 0.01145741 |
| Stone | CaOx | L-beta-aspartyl-L-aspartic acid | 283.06 | Negative | −2.1687725 | 0.01260603 |
| Stone | CaOx | Chondroitin | 378.11 | Negative | −2.1628256 | 0.03681497 |
| Stone | CaOx | 1,CPD-11674 5-hydroxytryptophol | 275.07 | Positive | −2.1384666 | 0.0398091 |
| | | sulfate | | | | |
| Stone | CaOx | 7,8-Dihydropteroic acid | 349.11 | Negative | −2.133229 | 0.00688045 |
| Stone | CaOx | Finasteridecarboxylic acid | 401.25 | Negative | −2.091432 | 0.04751673 |
| Stone | CaOx | Salidroside; p-Hydroxyphenethyl | 335.11 | Negative | −2.0692126 | 0.01049581 |
| | | alcohol 1-O-beta-D-glucoside; | | | | |
| | | Tyrosol glucoside | | | | |
| Stone | CaOx | L-Arginine, N2-[2-(2,2- | 411.21 | Negative | −2.0541939 | 0.0356559 |
| | | diphenylethoxy)acetyl | | | | |
| Stone | CaOx | Shikimate 3-phosphate; Shikimate | 253.01 | Negative | −2.0510506 | 0.01869302 |
| | | 5-phosphate | | | | |
| Stone | CaOx | 5-Androsten-3.beta.,17diol-3- | 369.18 | Negative | −2.0426545 | 0.00130001 |
| | | sulfate | | | | |
| Stone | CaOx | Methyl 18-bromo-15E,17E- | 399.1 | Negative | −2.0336396 | 0.02885561 |
| | | octadecadien-5,7-diynoate | | | | |
| Stone | CaOx | N-(Phenylacetyl)-L-phenylalanine | 282.11 | Negative | −2.0030957 | 0.04259391 |
| Stone | CaOx | Asp-Leu | 245.12 | Negative | −1.9886953 | 0.0311159 |
| Stone | CaOx | Biphenyl-2,3-diol; 2,3- | 185.06 | Negative | −1.9882397 | 0.03462426 |
| | | Dihydroxybiphenyl; 3- | | | | |
| | | Phenylcatechol | | | | |
| Stone | CaOx | n-Propyl gallate | 357.09 | Negative | −1.9754677 | 0.01379536 |
| Stone | CaOx | R-Dysidazirine | 325.28 | Positive | −1.933031 | 0.02981779 |
| Stone | CaOx | Asn-Asp | 246.08 | Negative | −1.9067652 | 0.04266881 |
| Stone | CaOx | C04133 N-Acetyl-L-glutamate 5- | 292.02 | Positive | −1.8850044 | 0.02646134 |
| | | phosphate | | | | |
| Stone | CaOx | Fraxin | 369.09 | Negative | −1.8760165 | 0.00882933 |
| Stone | CaOx | Galactinol dihydrate | 413.13 | Negative | −1.838872 | 0.04887481 |
| Stone | CaOx | 3-Cysteinylacetaminophen | 269.06 | Negative | −1.8362357 | 0.01126366 |
| Stone | CaOx | N-docosahexaenoyl glutamine | 479.29 | Positive | −1.7078241 | 0.03953972 |
| Stone | CaOx | 2-Aminomuconic acid | 159.08 | Positive | −1.6884404 | 0.01424012 |
| | | semialdehyde | | | | |
| Stone | CaOx | N-Acetylgalactosamine 4,6- | 178.06 | Negative | −1.6396621 | 0.01882369 |
| | | disulfate | | | | |
| Stone | CaOx | Oxaloglutarate | 239.02 | Negative | −1.6393396 | 0.00280063 |
| Stone | CaOx | Ala-Glu | 217.09 | Negative | −1.6289287 | 0.02067606 |
| Stone | CaOx | phorbol 13-acetate | 407.21 | Positive | −1.6242437 | 0.00163328 |
| Stone | CaOx | 5-Methyl-6-hydroxyluteolinidin | 321.12 | Positive | −1.6181205 | 0.03715814 |
| | | Flavonoids | | | | |
| Stone | CaOx | C20285 4-Acetylaminobiphenyl | 234.09 | Positive | −1.6103401 | 0.04214045 |
| Stone | CaOx | 5-Hydroxy-L-tryptophan | 221.09 | Positive | −1.600994 | 0.01206821 |
| Stone | CaOx | C00813 Barbiturate | 151.01 | Negative | −1.5902757 | 0.01253433 |
| Stone | CaOx | Glyoxylic acid | 72.99 | Negative | −1.5820907 | 0.03666101 |
| Stone | CaOx | Gamma Glutamylglutamic acid | 275.09 | Negative | −1.5461206 | 0.03025538 |
| Stone | CaOx | N1-(5-Phospho-alpha-D-ribosyl)- | 333.04 | Negative | −1.5319374 | 0.04504748 |
| | | 5,6-dimethylbenzimidazole; alpha- | | | | |
| | | Ribazole 5′-phosphate | | | | |
| Stone | CaOx | 4-Methylumbelliferyl D- | 351.07 | Negative | −1.5242352 | 0.04880857 |
| | | glucuronide | | | | |
| Stone | CaOx | 1-Pentanone, 1-(3,4- | 262.15 | Negative | −1.4883355 | 0.04835375 |
| | | dihydroxyphenyl)-2-(1- | | | | |
| | | pyrrolidinyl)- | | | | |
| Stone | CaOx | C18893 1-Methylseleno-N-acetyl- | 317.06 | Positive | −1.4792449 | 0.02698898 |
| | | D-galactosamine | | | | |
| Stone | CaOx | C07083 Styrene | 105.07 | Positive | −1.4695524 | 0.04166126 |
| Stone | CaOx | (S)-Reticuline | 330.17 | Positive | −1.4627729 | 0.03691803 |
| Stone | CaOx | Quercetin | 301.04 | Negative | −1.451958 | 0.01225654 |
| Stone | CaOx | 8-Hydroxyguanosine | 298.08 | Negative | −1.382623 | 0.00739829 |
| Stone | CaOx | DHAP(100) | 342.17 | Positive | −1.382135 | 0.01222423 |
| Stone | CaOx | C18325 Feruloylagmatine | 329.16 | Positive | −1.292835 | 9.33E−05 |
| Stone | CaOx | C05515 5-Ureido-4-imidazole | 188.08 | Positive | −1.2847959 | 0.03648216 |
| | | carboxylate | | | | |
| Stone | CaOx | 5-(Diphenylphosphinyl)pentanoic | 301.1 | Negative | −1.2749161 | 0.0269022 |
| | | acid | | | | |
| Stone | CaOx | 2-Amino-4-oxo-6-(1′,2′,3′- | 288.09 | Negative | −1.2689135 | 0.00267195 |
| | | trihydroxypropyl)-diquinoid-7,8- | | | | |
| | | dihydroxypterin | | | | |
| Stone | CaOx | C17245 5- | 148.08 | Positive | −1.2619164 | 0.00167811 |
| | | Methylthiopentanaldoxime | | | | |
| Stone | CaOx | Hydantoin-5-propionic acid | 171.04 | Negative | −1.255611 | 0.02750318 |
| Stone | CaOx | 1,CPD-4522 triparanol | 438.22 | Positive | −1.2516296 | 0.03121992 |
| Stone | CaOx | Imidazole | 103.03 | Negative | −1.2354669 | 0.00817041 |
| Stone | CaOx | Arachidonyl carnitine | 522.44 | Positive | −1.2329651 | 0.01942556 |
| Stone | CaOx | 3-Methoxy-4-hydroxyphenylglycol | 263.02 | Negative | −1.2252421 | 0.0413406 |
| | | sulfate | | | | |
| Stone | CaOx | Fexofenadine | 500.28 | Negative | −1.2126374 | 0.03875747 |
| Stone | CaOx | 2,3,6-Trihydroxypyridine | 126.02 | Negative | −1.2080965 | 0.0122422 |
| Stone | CaOx | 1,CPD0-2377 7-methylguanine | 167.08 | Positive | −1.2003042 | 0.02010619 |
| Stone | CaOx | Nitrilotriacetic acid | 147.09 | Positive | −1.1735062 | 0.03804607 |
| Stone | CaOx | 4-Hydroxynonenal | 155.11 | Negative | −1.091482 | 0.02313625 |
| Stone | CaOx | Pregnenolone sulfate | 431.19 | Negative | −1.077314 | 0.01872532 |
| Stone | CaOx | 9,13-cis-retinoate | 322.19 | Positive | −1.0624615 | 0.01507354 |
| Stone | CaOx | Salsoline-1-carboxylate | 235.08 | Negative | −0.9058105 | 0.00822744 |
| Stone | CaOx | 11′-carboxy-gamma-tocotrienol | 398.25 | Positive | −0.903461 | 0.04499912 |
| Stone | CaOx | C17232 2-Oxo-10- | 233.12 | Positive | −0.8285735 | 0.03333191 |
| | | methylthiodecanoic acid | | | | |
| Stone | CaOx | 3,5,7,9,11-dodecapentaenoic acid | 191.11 | Positive | −0.8130204 | 0.00261294 |
| Stone | CaOx | 1alpha-hydroxy-24- | 437.27 | Positive | −0.8116432 | 0.01281086 |
| | | methylsulfonyl-25,26,27- | | | | |
| | | trinorvitamin D3/1alpha-hydroxy- | | | | |
| | | 24-methylsulfonyl-25,26,27- | | | | |
| | | trinorcholecalciferol | | | | |
| Stone | CaOx | 1,CPD-4521 trans-1,4-bis(2- | 413.15 | Positive | −0.807227 | 0.04714751 |
| | | chlorobenzylaminomethyl)cyclo- | | | | |
| | | hexane dihydrochloride | | | | |
| Stone | CaOx | C20290 N-Acetoxy-IQ | 279.08 | Positive | −0.5330252 | 0.04778238 |
| Stone | CaOx | 2-(Trifluoromethyl)thiophenol | 177 | Negative | −0.4247388 | 0.00581618 |
| Stone | CaOx | Hemigossypol | 261.11 | Positive | −0.4191859 | 0.02711665 |
| Stone | CaPhos | 2-Amino-3-carboxymuconic acid | 184.03 | Negative | 0.4015765 | 0.02377618 |
| | | semialdehyde | | | | |
| Stone | CaPhos | 1,CPD0-1652 4-methylpyrazole | 83.06 | Positive | 0.42473062 | 0.01940507 |
| Stone | CaPhos | Aniline | 92.05 | Negative | 0.49675882 | 0.02032942 |
| Stone | CaPhos | (R)-Amphetamine | 136.11 | Positive | 0.52596216 | 0.03056551 |
| Stone | CaPhos | Dibenzo-p-dioxin | 219.05 | Negative | 0.53264709 | 0.01397552 |
| Stone | CaPhos | (−)-Caryophyllene oxide | 203.18 | Positive | 0.56619913 | 0.00802807 |
| Stone | CaPhos | 12,13-Dimethyl-5,14- | 237.15 | Positive | 0.58663353 | 0.02197629 |
| | | dioxabicyclo9.2.1-tetradeca- | | | | |
| | | 1(13),11-dien-4-one | | | | |
| Stone | CaPhos | 6,8-dihydroxy-octanoic acid | 177.11 | Positive | 0.58813565 | 0.02591209 |
| Stone | CaPhos | N-Isopropylammelide | 211.06 | Negative | 0.59411462 | 0.00714848 |
| Stone | CaPhos | C17010 de-Hypoxanthine | 319.08 | Positive | 0.60004945 | 0.01504727 |
| | | futalosine | | | | |
| Stone | CaPhos | Dopaxanthin | 389.1 | Negative | 0.61871215 | 0.04698473 |
| Stone | CaPhos | Methyl isopropyl ketone, 2,4- | 265.1 | Negative | 0.62040089 | 0.04420113 |
| | | dinitrophenylhydrazone | | | | |
| Stone | CaPhos | Hydroxyhexanoycarnitine | 274.17 | Negative | 0.62866754 | 0.02048111 |
| Stone | CaPhos | Postin | 531.31 | Negative | 0.66767176 | 0.04768513 |
| Stone | CaPhos | Pramanicin | 370.22 | Positive | 0.69650638 | 0.01306696 |
| Stone | CaPhos | Cyanosulfurous acid anion | 91.98 | Positive | 0.74497306 | 0.01405226 |
| Stone | CaPhos | 2-Hydroxydodecanoic acid | 215.17 | Negative | 0.76779468 | 0.00403594 |
| Stone | CaPhos | N-a-Acetylcitrulline | 252.1 | Negative | 0.77644682 | 0.03912751 |
| Stone | CaPhos | D-Glucuronic acid 1-phosphate | 273 | Negative | 0.78178846 | 0.0463543 |
| Stone | CaPhos | 2-pentylfuran | 139.11 | Positive | 0.78577575 | 0.01996275 |
| Stone | CaPhos | 2-Aminomuconic acid | 156.03 | Negative | 0.79120584 | 0.03146827 |
| Stone | CaPhos | Benzaldehyde | 124.08 | Positive | 0.83548812 | 0.04799808 |
| Stone | CaPhos | 2,5-Furandicarboxylic acid | 155 | Negative | 0.8568204 | 0.02509341 |
| Stone | CaPhos | 27-nor-campestan- | 517.34 | Positive | 0.87776111 | 0.03526824 |
| | | 3beta,4beta,5alpha,6alpha,7beta, | | | | |
| | | 8beta,14alpha,15alpha,24-nonol | | | | |
| Stone | CaPhos | 6,7-Dihydroxycoumarin-4-acetic | 235.02 | Negative | 0.87976963 | 0.01645342 |
| | | acid | | | | |
| Stone | CaPhos | 6-methyltetrahydropterin | 216.09 | Negative | 0.88849392 | 0.04935417 |
| Stone | CaPhos | Deoxyhypusine | 216.17 | Negative | 0.89099196 | 0.00429156 |
| Stone | CaPhos | L-3,4-Dihydroxybutan-2-one 4- | 219.01 | Negative | 0.89252554 | 0.00399543 |
| | | phosphate; 1-Deoxy-L-glycero- | | | | |
| | | tetrulose 4-phosphate; 2-Hydroxy- | | | | |
| | | 3-oxobutyl phosphate | | | | |
| Stone | CaPhos | C17 sphingosine-1- | 473.31 | Positive | 0.90132235 | 0.02728346 |
| | | phosphocholine | | | | |
| Stone | CaPhos | Dimethyl sulfoxide | 77.01 | Negative | 0.91006535 | 0.03924118 |
| Stone | CaPhos | N-Formyl-4-amino-5- | 205.07 | Positive | 0.92440488 | 0.02405111 |
| | | aminomethyl-2-methylpyrimidine; | | | | |
| | | 2-Methyl-4-amino-5- | | | | |
| | | (formylaminomethyl)pyrimidine; | | | | |
| | | FAMP | | | | |
| Stone | CaPhos | Cysteinylglycine | 213.03 | Negative | 0.93939023 | 0.01956446 |
| Stone | CaPhos | Leu-Gly-Gly | 244.13 | Negative | 0.95393653 | 0.01604198 |
| Stone | CaPhos | Dodecanedioic acid | 265.15 | Negative | 0.97273923 | 0.03884077 |
| Stone | CaPhos | 2-Hydroxyamino-1-methyl-6- | 239.1 | Negative | 0.98624419 | 0.0259705 |
| | | phenylimidazo[4,5-b | | | | |
| Stone | CaPhos | 3-Oxo-4,6-choladienoic acid | 405.24 | Negative | 0.99844969 | 0.00590871 |
| Stone | CaPhos | Carbamazepine-o-quinone | 301.06 | Negative | 1.0058728 | 0.0047687 |
| Stone | CaPhos | 2-Hexenoylcarnitine | 280.15 | Positive | 1.03327899 | 0.0439599 |
| Stone | CaPhos | 3-Furancarboxylic acid, | 253.15 | Negative | 1.03612377 | 0.01360919 |
| | | tetrahydro-4-methylene-2-octyl-5- | | | | |
| | | oxo-, (2R,3S)-rel- | | | | |
| Stone | CaPhos | Suberylglycine | 230.11 | Negative | 1.07435037 | 0.04855974 |
| Stone | CaPhos | 1,L-OCTANOYLCARNITINE L- | 311.21 | Positive | 1.11041932 | 0.02209854 |
| | | octanoylcarnitine | | | | |
| Stone | CaPhos | Ergothioneine | 229.09 | Negative | 1.11835491 | 0.00565501 |
| Stone | CaPhos | 1beta,3beta,5alpha,6beta- | 361.2 | Positive | 1.12182293 | 0.03728601 |
| | | tetrahydroxyandrostan-17-one | | | | |
| Stone | CaPhos | L-Pyrrolysine; Pyrrolysine | 254.15 | Negative | 1.15450878 | 0.00783991 |
| Stone | CaPhos | 3-Hydroxyisoheptanoic acid | 145.09 | Negative | 1.17704948 | 0.04386372 |
| Stone | CaPhos | Arg-Lys | 301.21 | Negative | 1.20616912 | 0.04169678 |
| Stone | CaPhos | 6-Methylmercaptopurine; | 201.02 | Negative | 1.21903037 | 0.01951674 |
| | | Thiopurine S-methylether | | | | |
| Stone | CaPhos | Dodecanoic acid | 199.17 | Positive | 1.32476489 | 0.02683987 |
| Stone | CaPhos | Arachidonoylmorpholine | 391.33 | Positive | 1.35940393 | 0.00785903 |
| Stone | CaPhos | 1,11-Undecanedicarboxylic acid | 243.16 | Negative | 1.38235644 | 0.00909129 |
| Stone | CaPhos | C13632 4,4′-Dihydroxy-alpha- | 227.11 | Positive | 1.38703551 | 0.00092576 |
| | | methylstilbene | | | | |
| Stone | CaPhos | 2-Keto-6-acetamidocaproate | 186.08 | Negative | 1.38931581 | 0.01610225 |
| Stone | CaPhos | Sulfoacetate; Sulfoacetic acid | 138.97 | Negative | 1.42791125 | 0.01524446 |
| Stone | CaPhos | 26,26,26-trifluoro-25-hydroxy-27- | 463.28 | Positive | 1.42807375 | 0.04658581 |
| | | norvitamin D3/26,26,26- | | | | |
| | | trifluoro-25-hydroxy-27- | | | | |
| | | norcholecalciferol | | | | |
| Stone | CaPhos | C16264 2-Hydroxy-6-oxo-6-(2- | 263.06 | Positive | 1.43119204 | 0.00077991 |
| | | carboxyphenyl)-hexa-2,4-dienoate | | | | |
| Stone | CaPhos | MeIQx; 8-Methyl-IQX; 2-Amino- | 212.09 | Negative | 1.44686652 | 0.00098477 |
| | | 3,8-dimethylimidazo[4,5-f | | | | |
| Stone | CaPhos | Carbidopa | 225.09 | Negative | 1.46691648 | 0.01808461 |
| Stone | CaPhos | C07493 10-Hydroxycarbazepine | 277.09 | Positive | 1.50297525 | 0.02393275 |
| Stone | CaPhos | N-(2-fluro-ethyl) 2-methyl- | 386.28 | Positive | 1.50456317 | 0.00052677 |
| | | arachidonoyl amine | | | | |
| Stone | CaPhos | 2-[2-(2-Methoxyethoxy)ethoxy | 177.08 | Negative | 1.55985645 | 6.42E−05 |
| Stone | CaPhos | 4-Guanidinobutanoic acid | 144.08 | Negative | 1.5749377 | 0.00063113 |
| Stone | CaPhos | Phe-His | 301.13 | Negative | 1.58253514 | 0.00397623 |
| Stone | CaPhos | N-Acetyl-D-glucosamine | 202.07 | Negative | 1.59602277 | 0.0108002 |
| Stone | CaPhos | 5-Sulfosalicylic acid | 252.98 | Negative | 1.61495863 | 0.03843382 |
| Stone | CaPhos | Bisnorcholic acid | 403.24 | Positive | 1.6217688 | 0.01635324 |
| Stone | CaPhos | 7,17Dimethyl-5androstane- | 495.3 | Negative | 1.63165075 | 0.01421171 |
| | | 3,17diol glucuronide | | | | |
| Stone | CaPhos | 6-Thiourate; 6-Thiouric acid | 219 | Negative | 1.68010539 | 0.00170126 |
| Stone | CaPhos | Thr-Met | 249.09 | Negative | 1.68950229 | 0.04622745 |
| Stone | CaPhos | 2-Pentenoic acid, 5-(4- | 285.06 | Negative | 1.72126837 | 0.00115239 |
| | | chlorophenyl)-3-phenyl-, (2Z)- | | | | |
| Stone | CaPhos | formyl 7-oxo-11E-tetradecenoate | 255.2 | Positive | 1.80386789 | 0.02750681 |
| Stone | CaPhos | Vomitoxin Isoprenoids | 314.16 | Positive | 1.8098707 | 0.04237386 |
| Stone | CaPhos | Arbutin; Ursin; Uvasol; | 307.08 | Negative | 1.82311286 | 0.00185737 |
| | | Hydroquinone-O-beta-D- | | | | |
| | | glucopyranoside | | | | |
| Stone | CaPhos | 7,8-dichloro-hexadecanoic acid | 359.15 | Negative | 1.82844461 | 3.19E−05 |
| Stone | CaPhos | Hesperetin | 301.07 | Negative | 1.83664828 | 0.00593441 |
| Stone | CaPhos | Sphingosine-1-phosphocholine | 473.31 | Negative | 1.92809187 | 0.00883682 |
| | | Sphingoid bases | | | | |
| Stone | CaPhos | 11-dehydro-2,3-dinor-TXB2 | 358.22 | Positive | 1.94900489 | 0.01460277 |
| Stone | CaPhos | Allantoic acid | 175.05 | Negative | 1.95938029 | 0.00050168 |
| Stone | CaPhos | (11Z)-8,18-methano-retinal | 331.21 | Negative | 1.99686201 | 0.02351026 |
| | | Isoprenoids | | | | |
| Stone | CaPhos | 18-fluoro-9Z,12Z-octadecadienoic | 299.24 | Positive | 2.01149924 | 0.04647668 |
| | | acid | | | | |
| Stone | CaPhos | Sulfometuron methyl | 363.08 | Negative | 2.01223992 | 0.04374018 |
| Stone | CaPhos | 3S-bromobutanoic acid | 200.96 | Negative | 2.02610068 | 0.01818482 |
| Stone | CaPhos | Rosiglitazone | 356.11 | Negative | 2.06209074 | 0.04044841 |
| Stone | CaPhos | N-Acetylisoniazid; (N)1- | 201.08 | Negative | 2.06697703 | 0.04622015 |
| | | Acetylisoniazid | | | | |
| Stone | CaPhos | Pipemidic acid | 302.13 | Negative | 2.12866999 | 0.04056986 |
| Stone | CaPhos | Nicotinate D-ribonucleoside | 179.05 | Negative | 2.13450673 | 0.04212879 |
| Stone | CaPhos | Thiourocanic acid | 205.01 | Negative | 2.14041911 | 0.03528605 |
| Stone | CaPhos | 5,6-Dihydro-5-fluorouracil; 5- | 167.03 | Negative | 2.15087481 | 0.04098494 |
| | | Fluorodihydrouracil | | | | |
| Stone | CaPhos | Nornicotine | 147.09 | Negative | 2.17686981 | 0.04071115 |
| Stone | CaPhos | Vitamin K1 | 485.34 | Negative | 2.19492529 | 0.03233129 |
| Stone | CaPhos | Diacetylrhein | 367.05 | Negative | 2.20332778 | 0.0149508 |
| Stone | CaPhos | Brosimacutin C | 343.15 | Positive | 2.22778069 | 0.01768882 |
| Stone | CaPhos | 3alpha,7alpha,12alpha-trihydroxy- | 475.27 | Positive | 2.24661724 | 0.04914608 |
| | | 5alpha-cholan-24-yl sulfate | | | | |
| Stone | CaPhos | 5′-Butyrylphosphoinosine; Inosine | 453.08 | Negative | 2.26887241 | 0.03583696 |
| | | 5′-phosphobutyrate | | | | |
| Stone | CaPhos | L-Thyronine | 272.09 | Negative | 2.34920301 | 0.0197003 |
| Stone | CaPhos | Tegafur | 199.05 | Negative | 2.35132311 | 0.02930195 |
| Stone | CaPhos | 3b-Hydroxy-5-cholenoic acid | 375.29 | Positive | 2.3520785 | 0.02267718 |
| Stone | CaPhos | 11-bromo-undecanoic acid | 299.07 | Negative | 2.38981202 | 0.00325159 |
| Stone | CaPhos | 3-Sulfino-L-alanine; L- | 152 | Negative | 2.39999348 | 0.00038787 |
| | | Cysteinesulfinic acid; 3-Sulphino-L- | | | | |
| | | alanine; 3-Sulfinoalanine | | | | |
| Stone | CaPhos | Oleandolide Macrolides and | 387.24 | Positive | 2.40855104 | 0.00420916 |
| | | lactone polyketides | | | | |
| Stone | CaPhos | Urea | 95.03 | Negative | 2.42457499 | 0.03435231 |
| Stone | CaPhos | Calomelanol D-1 | 406.17 | Positive | 2.43092658 | 0.04983077 |
| Stone | CaPhos | 2,2′,3-Trihydroxybiphenyl | 201.06 | Negative | 2.4313601 | 0.00010024 |
| Stone | CaPhos | 13S-Hydroxy-9Z,11E- | 295.23 | Negative | 2.4453332 | 2.06E−05 |
| | | octadecadienoic acid | | | | |
| Stone | CaPhos | 10Z,13Z,16Z-nonadecatrienenitrile | 296.23 | Positive | 2.45695795 | 0.00697925 |
| Stone | CaPhos | Lorazepam | 319.01 | Negative | 2.498909 | 0.02101402 |
| Stone | CaPhos | LysoSM(d18:1) | 500.34 | Negative | 2.50346683 | 0.01956395 |
| Stone | CaPhos | Cervonoyl ethanolamide | 373.27 | Positive | 2.52980185 | 0.03384924 |
| Stone | CaPhos | OH-Demethylspheroidenone | 604.47 | Positive | 2.53790496 | 0.01817425 |
| Stone | CaPhos | C03621 N-Acetoxy-4- | 245.13 | Positive | 2.54541091 | 0.00227149 |
| | | aminobiphenyl | | | | |
| Stone | CaPhos | 13,16-Docosadienoic acid; | 371.3 | Negative | 2.57554612 | 0.02819584 |
| | | (13Z,16Z)-Docosa-13,16-dienoic | | | | |
| | | acid | | | | |
| Stone | CaPhos | 2-Oxo-9-methylthiononanoic acid; | 253.09 | Negative | 2.5910451 | 0.00879248 |
| | | 9-Methylthio-2-nonanoic acid | | | | |
| Stone | CaPhos | 4-Dedimethylamine-4-oxo- | 466.03 | Negative | 2.61848606 | 0.00975008 |
| | | anhydro-7-Cl-tetracycline Linear | | | | |
| | | tetracyclines | | | | |
| Stone | CaPhos | C03458 2,3,6-Trihydroxypyridine | 145.06 | Positive | 2.6530619 | 0.00539773 |
| Stone | CaPhos | Cyanuric acid | 128.01 | Negative | 2.65571677 | 0.004853 |
| Stone | CaPhos | bromovulone I | 459.12 | Negative | 2.65916554 | 0.00608761 |
| Stone | CaPhos | 5-hydroxytryptophol glucuronide | 338.13 | Negative | 2.66551547 | 0.03898021 |
| Stone | CaPhos | 5-Amino-6-(5′- | 353.05 | Negative | 2.68036946 | 0.00056375 |
| | | phosphoribosylamino)uracil; 5- | | | | |
| | | Amino-6-(ribosylamino)-2,4- | | | | |
| | | (1H,3H)-pyrimidinedione 5′- | | | | |
| | | phosphate; 5-Amino-6-(5- | | | | |
| | | phosphoribosylamino)uracil | | | | |
| Stone | CaPhos | troglitazone | 440.15 | Negative | 2.70734921 | 0.01024857 |
| Stone | CaPhos | 2-Arachidonyl Glycerol ether | 382.33 | Positive | 2.70735295 | 0.01260058 |
| Stone | CaPhos | Met-Gly-Met | 336.11 | Negative | 2.71590518 | 0.00041628 |
| Stone | CaPhos | 3′-Ketolactose | 339.09 | Negative | 2.74371662 | 0.00933353 |
| Stone | CaPhos | 1-Fluoro-25-hydroxy-16-ene-23- | 419.32 | Positive | 2.76058817 | 0.00633373 |
| | | yne-26,27-hexadeuterovitamin-D3 | | | | |
| Stone | CaPhos | 3-oxo-hexadecanoyl-acp | 308.18 | Positive | 2.77141893 | 0.01640188 |
| Stone | CaPhos | 1alpha,25-dihydroxy-25,25- | 563.35 | Positive | 2.82086229 | 0.01099463 |
| | | diphenyl-26,27-dinorvitamin D3/ | | | | |
| | | 1alpha,25-dihydroxy-25,25- | | | | |
| | | diphenyl-26,27- | | | | |
| | | dinorcholecalciferol | | | | |
| Stone | CaPhos | 3,4-Dihydroxybenzylamine | 157.1 | Positive | 2.84222388 | 0.02054805 |
| Stone | CaPhos | Vinyl chloride; Chloroethylene | 96.99 | Negative | 2.8737688 | 0.02272937 |
| Stone | CaPhos | (2R,3R)-3-Methylglutamyl-5- | 272.16 | Positive | 2.87473128 | 0.01611797 |
| | | semialdehyde-N6-lysine | | | | |
| Stone | CaPhos | Fluoren-9-one; 9-Fluorenone | 215.05 | Negative | 2.87731122 | 0.00642163 |
| Stone | CaPhos | Phe-Tyr | 327.13 | Negative | 2.88253665 | 0.04114357 |
| Stone | CaPhos | Pyridoxamine 5′-phosphate | 283.05 | Negative | 2.88505765 | 0.00568483 |
| Stone | CaPhos | PS(203(8Z,11Z,14Z)/00) | 565.33 | Positive | 2.91925071 | 0.0122626 |
| Stone | CaPhos | Palmitoyl glucuronide | 453.29 | Negative | 2.95907577 | 0.02059869 |
| Stone | CaPhos | Feruloylputrescine; Subaphyllin | 299.14 | Negative | 2.96305403 | 0.01176559 |
| Stone | CaPhos | 3-Oxooctadecanoic acid | 321.24 | Positive | 2.96974987 | 1.25E−07 |
| Stone | CaPhos | Leucettamol A | 495.39 | Positive | 3.00560778 | 0.00725028 |
| Stone | CaPhos | trans, trans-Farnesyl phosphate; | 301.16 | Negative | 3.01075493 | 0.01924084 |
| | | Farnesyl phosphate; (2E,6E)- | | | | |
| | | Farnesyl phosphate | | | | |
| Stone | CaPhos | Aflatoxin B1 | 347.05 | Negative | 3.02077617 | 0.00630587 |
| Stone | CaPhos | Styrene glycol, glucuronide | 313.1 | Negative | 3.02300984 | 0.00623 |
| Stone | CaPhos | C01433 Loganin | 413.14 | Positive | 3.0714145 | 0.0064845 |
| Stone | CaPhos | PC(O-181(9Z)/O-10) | 539.42 | Positive | 3.07279653 | 0.00687256 |
| Stone | CaPhos | Desomorphine | 270.15 | Negative | 3.07557557 | 0.00042721 |
| Stone | CaPhos | C00296 Quinate | 210.1 | Positive | 3.17974667 | 0.00643419 |
| Stone | CaPhos | Gossypetin 3-O-sulfate Flavonoids | 331.05 | Negative | 3.18103241 | 0.00443004 |
| Stone | CaPhos | Tenofovir | 286.07 | Negative | 3.19128066 | 4.72E−05 |
| Stone | CaPhos | PG(190/00) | 549.32 | Positive | 3.19692562 | 0.00980095 |
| Stone | CaPhos | O-Desmethylangolensin | 293.08 | Negative | 3.21200399 | 0.0013359 |
| Stone | CaPhos | Methylimidazoleacetic acid; Tele- | 175.05 | Negative | 3.28544208 | 0.00580189 |
| | | methylimidazoleacetic acid; 1- | | | | |
| | | Methyl-4-imidazoleacetic acid; 1- | | | | |
| | | Methylimidazole-4-acetate; | | | | |
| | | Methylimidazoleacetate | | | | |
| Stone | CaPhos | 3-Bromotyrosine | 293.98 | Negative | 3.28612774 | 0.00496042 |
| Stone | CaPhos | D-Erythro-imidazole-glycerol- | 237.03 | Negative | 3.29104583 | 0.00019972 |
| | | phosphate | | | | |
| Stone | CaPhos | 6-Mercaptopurine; | 151.01 | Positive | 3.31474046 | 0.00155341 |
| | | Mercaptopurine | | | | |
| Stone | CaPhos | nicotinamide mononucleotide | 255.07 | Negative | 3.33683507 | 0.00207918 |
| Stone | CaPhos | 6-Methoxynaphthaleneacetic acid | 215.08 | Negative | 3.34023172 | 0.0010596 |
| Stone | CaPhos | (−)-Fusicoplagin A Isoprenoids | 439.27 | Positive | 3.34879311 | 0.00263213 |
| Stone | CaPhos | Phosphoribosyl | 365.05 | Negative | 3.38160324 | 0.00083878 |
| | | formamidocarboxamide | | | | |
| Stone | CaPhos | Pelargonidin 3-(2′- | 639.22 | Positive | 3.41092622 | 0.00189416 |
| | | acetylrutinoside) | | | | |
| Stone | CaPhos | DMG-MINO Linear tetracyclines | 592.24 | Negative | 3.4247325 | 0.00326252 |
| Stone | CaPhos | Flumequine | 260.07 | Negative | 3.47120531 | 0.00194953 |
| Stone | CaPhos | Salicin 6-phosphate; Salicin-6P | 365.06 | Negative | 3.5075983 | 0.00831492 |
| Stone | CaPhos | Sucralose | 395.01 | Negative | 3.53362915 | 0.00291835 |
| Stone | CaPhos | N-(2,4-Dinitrophenyl)-L-valine | 282.07 | Negative | 3.54640604 | 0.00027649 |
| Stone | CaPhos | 11-Oxo-androsterone glucuronide | 479.23 | Negative | 3.55349504 | 0.00273743 |
| Stone | CaPhos | Pantothenol | 204.13 | Negative | 3.5603926 | 0.00152482 |
| Stone | CaPhos | 3-Oxostearic acid | 297.25 | Negative | 3.67399166 | 8.08E−08 |
| Stone | CaPhos | Methanone, [1-(5-fluoropentyl)-2- | 399.17 | Negative | 3.70001439 | 3.47E−05 |
| | | hydroxy-1H-indol-3-yl | | | | |
| Stone | CaPhos | 3-[(3aS,4S,7aS)-7a-Methyl-1,5- | 273.11 | Negative | 3.80445368 | 0.0038024 |
| | | dioxo-octahydro-1H-inden-4-yl | | | | |
| Stone | CaPhos | D-Lombricine | 269.07 | Negative | 3.82397342 | 8.60E−05 |
| Stone | CaPhos | N-(2-fluro-ethyl) arachidonoyl | 384.27 | Negative | 3.8340166 | 0.00015581 |
| | | amine | | | | |
| Stone | CaPhos | (S)-Ureidoglycine | 132.04 | Negative | 3.86403448 | 0.00051388 |
| Stone | CaPhos | Eucalyptin Flavonoids | 327.12 | Positive | 3.94076294 | 0.00162977 |
| Stone | CaPhos | Ethyl hydrogen | 123.02 | Negative | 3.94239975 | 0.00014012 |
| | | methylphosphonate | | | | |
| Stone | CaPhos | Androstenedione | 321.19 | Negative | 3.98821573 | 0.00124168 |
| Stone | CaPhos | (−)-Asbestinine 2 Isoprenoids | 475.31 | Negative | 3.99527307 | 0.00038668 |
| Stone | CaPhos | 2-Hydroxypalmitic acid | 271.23 | Positive | 4.03007341 | 6.05E−06 |
| Stone | CaPhos | dolichyl diphosphate | 343.11 | Negative | 4.07408471 | 0.0004378 |
| Stone | CaPhos | 5beta-cholestan- | 613.27 | Positive | 4.15634384 | 2.24E−05 |
| | | 3alpha,4alpha,11beta,12beta,21- | | | | |
| | | pentol-3,21-disulphate | | | | |
| Stone | CaPhos | L-Serine-phosphoethanolamine; | 227.04 | Negative | 4.19650081 | 9.78E−05 |
| | | Serine phosphoethanolamine | | | | |
| Stone | CaPhos | C07495 Dihydroxycarbazepine | 288.13 | Positive | 4.22409239 | 3.56E−05 |
| Stone | CaPhos | 16-hydroxy hexadecanoic acid | 294.22 | Positive | 4.29502445 | 0.00094113 |
| Stone | CaPhos | Desulfoglucotropeolin | 364.09 | Negative | 4.32099851 | 0.00024119 |
| Stone | CaPhos | N-Acetyl-D-lactosamine | 380 | Negative | 4.42579308 | 0.00017584 |
| Stone | CaPhos | L-Homocysteic acid | 182.01 | Negative | 4.504363 | 0.00067075 |
| Stone | CaPhos | Malonylcarnitine | 246.1 | Negative | 4.6532457 | 2.44E−05 |
| Stone | CaPhos | 5-Methyldeoxycytidine | 240.1 | Negative | 4.75693483 | 1.49E−05 |
| Stone | CaPhos | epsilon-Caprolactam | 112.08 | Negative | 4.84392754 | 2.38E−06 |
| Stone | CaPhos | 12-chloro-dodecanoic acid | 257.13 | Positive | 4.98479786 | 2.24E−05 |
| Stone | CaPhos | C16362 3,6,8-Trimethylallantoin | 223.08 | Positive | 5.41604456 | 5.79E−06 |
| Stone | CaPhos | 3-Hydroxyoctadecanoic acid | 299.26 | Negative | 5.42571844 | 8.17E−09 |
| Stone | CaPhos | 3-Hydroxymyristic acid | 243.2 | Negative | 5.53093444 | 1.32E−08 |
| Stone | CaPhos | S-Carboxymethyl-L-cysteine | 178.02 | Negative | 5.64943664 | 9.72E−10 |
| Stone | CaPhos | 10-Hydroxyloganin Isoprenoids | 441.14 | Negative | 5.81075235 | 5.77E−09 |
| Stone | CaPhos | Sulfolithocholic acid | 455.25 | Negative | 5.86435223 | 2.24E−07 |
| Stone | CaPhos | PA(171(9Z)/00) | 445.23 | Positive | 5.95806677 | 5.43E−07 |
| Stone | CaPhos | Distemonanthin | 359.04 | Positive | 5.99198161 | 2.17E−09 |
| Stone | CaPhos | 1-Fluoro-25-hydroxy-16-ene-23- | 419.32 | Positive | 6.46507327 | 1.59E−09 |
| | | yne-26,27-hexadeuterovitamin-D3 | | | | |
| Stone | CaPhos | PI(P-200/00) | 630.4 | Positive | 6.5472113 | 1.02E−09 |
| Stone | CaPhos | 2,3-Dinor-8-iso prostaglandin | 325.2 | Negative | 6.66057774 | 1.23E−09 |
| | | F2alpha; 2,3-Dinor-8-iso | | | | |
| | | PGF2alpha | | | | |
| Stone | CaPhos | Estradiol valerate Steroids | 374.27 | Positive | 7.00925651 | 2.04E−08 |
| Stone | CaPhos | 5-Oxo-ETE-d7 | 343.3 | Positive | 7.05926395 | 5.31E−11 |
| Stone | CaPhos | 2-Methyl-4-amino-5- | 334 | Negative | 7.61104882 | 4.41E−17 |
| | | hydroxymethylpyrimidine | | | | |
| | | diphosphate; 4-Amino-2-methyl-5- | | | | |
| | | diphosphomethylpyrimidine | | | | |
| Stone | CaPhos | 4-Methylthiobutylthiohydroximate | 200.02 | Negative | 7.82070328 | 7.28E−18 |
| Stone | CaPhos | Thiamine aldehyde; Thiamin | 262.09 | Negative | 7.88969916 | 8.35E−11 |
| | | aldehyde | | | | |
| Stone | CaPhos | 3-Oxotetradecanoic acid | 265.18 | Positive | 8.43573834 | 2.03E−13 |
| Stone | CaPhos | Cystathionine sulfoxide | 256.1 | Positive | 9.06568338 | 2.91E−21 |
| Stone | CaPhos | (35,4S)-3-hydroxytetradecane- | 272.16 | Negative | 9.07796126 | 2.13E−27 |
| | | 1,3,4-tricarboxylic acid | | | | |
| Stone | CaPhos | 3-Oxohexadecanoic acid | 271.23 | Negative | 9.7685176 | 2.68E−25 |
| Stone | CaPhos | C04332 6,7-Dimethyl-8-(D- | 344.16 | Positive | 10.126023 | 1.12E−20 |
| | | ribityl)lumazine | | | | |
| Stone | CaPhos | Docosahexaenoic acid | 329.25 | Positive | 10.2089805 | 2.06E−19 |
| Urine | NonActive | Pyrithiobac | 327.01 | Positive | 12.401319 | 0.03710287 |
| Urine | NonActive | Valsartan | 458.21 | Positive | 9.2831693 | 0.03576064 |
| Urine | NonActive | LMPR0102070010: Harpagoside | −493.17 | Negative | 7.9714817 | 0.02373001 |
| | | Isoprenoids [PR01 | | | | |
| Urine | NonActive | 1,BETAINE glycine betaine | 136.12 | Positive | 4.7915301 | 0.02373001 |
| Urine | NonActive | Mitoxantrone | 445.21 | Positive | 4.5984612 | 0.03710287 |
| Urine | NonActive | trans-Ferulic acid | −193.05 | Negative | 3.6738917 | 0.00717055 |
| Urine | NonActive | 1-(2-Pyrimidyl)piperazine | 165.11 | Positive | 3.3841503 | 0.00275656 |
| Urine | NonActive | 1-Piperidinepropanenitrile, 4- | 335.16 | Positive | 3.1724297 | 0.00711786 |
| | | methyl-3-(methyl-7H-pyrrolo2,3- | | | | |
| | | dpyrimidin-4-ylamino)-oxo-, | | | | |
| | | (3R,4R)- | | | | |
| Urine | NonActive | 1-Pyrrolidineheptanoic acid, 2-(3- | 342.35 | Positive | 2.3803239 | 0.00461829 |
| | | hydroxyoctyl)-5-oxo- M + H − H2O+> | | | | |
| Urine | NonActive | 4,8-Dimethyl-1,3E,7-nonatriene | 151.15 | Positive | 2.348987 | 0.04344108 |
| Urine | NonActive | Solanidine | 398.34 | Positive | 2.2503979 | 0.03007526 |
| Urine | NonActive | D-myo-Inositol-2,4-diphosphate | −338.99 | Negative | 2.2358464 | 0.04441385 |
| Urine | NonActive | 1/1,[Cer(d18:0/16:0)] | 540.35 | Positive | 1.7646879 | 0.00650699 |
| Urine | NonActive | 4-Hydroxy-2-oxoglutaric acid | −161.01 | Negative | 1.6631922 | 0.03007526 |
| Urine | NonActive | 7-Hydroxycoumarin-4-acetic acid | −219.03 | Negative | 1.5838392 | 0.00668192 |
| Urine | NonActive | 1,2-Benzenediamine, 4-methyl- | 214.18 | Positive | 1.5813825 | 0.01668199 |
| | | N1-(3-phenylpropyl)- M + H+> | | | | |
| Urine | NonActive | 5,8-Tetradecadienoic acid | 225.19 | Positive | 1.5280266 | 0.04842123 |
| Urine | NonActive | 6E-Nonatrienal | 137.1 | Positive | 1.4192422 | 0.00650699 |
| Urine | NonActive | 2,3,6-Trihydroxypyridine | −126.02 | Negative | 1.3347339 | 0.00163434 |
| Urine | NonActive | ammelide | −163.03 | Negative | 1.3200561 | 0.04332883 |
| Urine | NonActive | Crocetin | 329.17 | Positive | 1.3081357 | 0.00275656 |
| Urine | NonActive | 18-Carboxy-dinor-LTE4 | 457.2 | Positive | 1.2942004 | 0.00134268 |
| Urine | NonActive | Hydroxybutyrylcarnitine | −246.14 | Negative | 1.2637551 | 0.0300375 |
| Urine | NonActive | Desulfoglucotropeolin | 374.13 | Positive | 1.2385499 | 0.01854201 |
| Urine | NonActive | Cyclohexane-1,2,4,5- | −259.05 | Negative | 1.2027868 | 0.04179149 |
| | | tetracarboxylic acid | | | | |
| Urine | NonActive | Dodecanoic acid | −199.17 | Negative | 1.1972665 | 0.03007526 |
| Urine | NonActive | 1,CPD-7664 tolcapone | 292.1 | Positive | 1.176649 | 0.00275656 |
| Urine | NonActive | 1-Propanol | 61.07 | Positive | 1.1588121 | 0.00711786 |
| Urine | NonActive | 5-Androstene-3b,16b,17a-triol | 307.22 | Positive | 1.1436666 | 0.01668199 |
| Urine | NonActive | (1-(5-Fluoropentyl)-1H-indazole-3- | 332.18 | Positive | 1.116676 | 0.04938897 |
| | | carbonyl)-L-valine | | | | |
| Urine | NonActive | 3,5,7-Trimethyl-2E,4E,6E,8E- | 177.16 | Positive | 1.1095265 | 0.00134268 |
| | | decatetraene | | | | |
| Urine | NonActive | C15970 Abscisic acid glucose ester | 427.2 | Positive | 1.0793488 | 0.04292705 |
| Urine | NonActive | Triptophenolide | −311.17 | Negative | 1.0649686 | 0.02281271 |
| Urine | NonActive | Androstenedione | −285.19 | Negative | 1.0503178 | 0.0340383 |
| Urine | NonActive | Cytidine 2′,3′-cyclic phosphate | 328.03 | Positive | 1.01682523 | 0.00644919 |
| Urine | NonActive | 1/1,[Uric acid] | 167.02 | Negative | 1.06087993 | 0.01023406 |
| Urine | NonActive | 1-Methyl-4-phenyl-1,2,3,6- | 174.73 | Positive | 1.1596802 | 0.03173347 |
| | | tetrahydropyridine | | | | |
| Urine | NonActive | Betaine | 118.08 | Positive | 1.19651613 | 0.04332883 |
| Urine | NonActive | 1,12E-Pentadecadiene | 228.27 | Positive | 1.19968203 | 0.01668199 |
| Urine | NonActive | 1,1-CHLORO-24-DINITROBENZENE | 205 | Positive | 1.28858184 | 0.00031008 |
| | | 1-chloro-2,4-dinitrobenzene | | | | |
| Urine | NonActive | Succinylacetone | 159.07 | Positive | 1.35853149 | 0.03943456 |
| Urine | NonActive | Inodxyl glucuronide | 332.07 | Positive | 1.86245113 | 0.01061191 |
| Urine | NonActive | 2-Methoxyestrone | 318.21 | Positive | 2.16888686 | 0.03710287 |
| Urine | NonActive | 22-tricosenoic acid | 353.34 | Positive | 2.49842788 | 0.03710287 |
| Urine | NonActive | Glycerophosphoglycerols | 698.5 | Positive | 5.00250365 | 0.03710287 |
| Urine | NonActive | Prostaglandin | 444.3 | Positive | 6.24939201 | 0.03329703 |
| Urine | NonActive | 1/1,[Pantothenol] | 206.14 | Positive | 7.56627731 | 0.03710287 |
| Urine | NonActive | Glycerophosphoglycerols | 698.5 | Positive | 9.19729481 | 0.03710287 |
| Urine | NonActive | N-Hydroxy-4-aminobiphenyl | 186.09 | Positive | 13.7694504 | 0.03710287 |

1. A composition, kit, or system comprising:
a) a sample from a subject having: urinary stone disease, or who is suspected of having urinary stone disease, or who has a recurrent episode of urinary stone disease, and
b) at least two stable isotope labeled urine metabolites selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether.

2. The composition, kit, or system of claim 1, wherein said stable isotope is selected from ²H, ¹³C, and ¹⁵N.

3. The composition, kit, or system of claim 1, wherein said sample comprises a urine sample.

4. The composition, kit, or system of claim 1, further comprising: c) un-labelled versions of said at least two urine metabolites.

5. The composition, kit, or system of claim 1, wherein said sample further comprises a stable isotope labeled third, fourth, fifth, sixth, seventh, eighth, ninth, or tenth stable isotope labeled urinary metabolite from the recited group.

6. A method of performing an activity based on a level of at least two urine metabolites in a urine sample from a subject comprising:
a) determining, or receiving information regarding, the level of said at least two urine metabolites in, or from, a urine sample from a subject,
wherein said at least two urine metabolites are selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether; and
b) identifying an increased level of said at least two urine metabolites in said sample compared to corresponding control values, and performing at least one of the following activities:
i) imaging the urinary tract of said subject and generating an image that shows the size and/or number and/or position of at least one urinary stone;
ii) treating said subject with an agent or procedure that treats urinary stone disease;
iii) generating and/or transmitting a report that: A) displays the level of said at least two urinary metabolites, and B) indicates that said subject is in need of: A) said imaging, and/or B) said agent or procedure that treat urinary stone disease; and
iv) characterizing said subject as having urinary stones, recurrence of urinary stones, urinary stone disease, recurrence of urinary stone disease, having recurrent stone activity, or an elevated risk for urinary stones or urinary stone disease.

7. The method of claim 6, wherein said at least two urine metabolites is at least three of said urine metabolites.

8. The method of claim 7, wherein said at least three urine metabolites is at least four of said urine metabolites.

9. The method of claim 6, wherein: A) wherein said imaging is selected from: computed tomography (CT), ultrasound, x-ray, and KUB x-ray; and/or B) said urinary stone disease therapeutic is selected from: thiazide, a potassium supplement, a magnesium supplement, and a calcium supplement; and/or C) wherein said procedure comprises removal of at least one urinary stone from said subject.

10. The method of claim 6, wherein said subject has, or is suspected of having, urinary stone disease, or wherein said subject has, or is suspected of having, recurrent urinary stone disease.

11. The method of claim 6, wherein said determining the level comprises the use of mass spectrometry with chromatography.

12. A method of treatment and/or imaging comprising:
a) identifying a subject as having increased levels of at least two urinary metabolites compared to corresponding reference values, wherein said at least two urinary metabolites are selected from: butanal, 2-Hydroxy-1,3-dimethoxy-8,9-methylenedioxycoumestan, 6-Methylmercaptopurine, Dimethyl-L-arginine, N-butanoyl-lhomoserine lactone, Hexanoylglycine, Methyl propenyl ketone, Ferulate, 2-Oxoarginine, and 2-Hydroxyestradiol-3-methyl ether; and
b) performing at least one of the following activities: i) treating said subject with a therapeutic or procedure that treats urinary stone disease, and/or ii) imaging the urinary tract of said subject and generating an image that shows the size and/or number and/or position of at least one urinary stone, and optionally generating a written plan of care tailored to said size, number, and/or position of said at least one urinary stone.

13. The method of claim 12, wherein said at least two urine metabolites is at least three of said urine metabolites.

14. The method of claim 12, wherein: i) said urinary stone disease therapeutic is selected from: thiazide, a potassium supplement, a magnesium supplement, and a calcium supplement, and ii) wherein said written plan comprises instructions for therapeutic treatment, expulsive therapy, and/or surgical intervention to remove said at least one urinary stone., and/or iii) said imaging is selected from: computed tomography (CT), ultrasound, x-ray, and KUB x-ray.

15. The method of claim 12, wherein said subject: i) has, or is suspected of having, urinary stone disease, has recurrence of urinary stones, iii) has recurrence of urinary stone disease, iv) has recurrent stone activity, or v) has an elevated risk for urinary stones or urinary stone disease.

16-20. (canceled)
